(19) United States
(12) Patent Application Publication
(10) Pub. No.: US 2015/0284420 A1
(43) Pub. Date: Oct. 8, 2015

(54) E-SELECTIN ANTAGONIST COMPOUNDS AND METHODS OF USE

(71) Applicant: GLYCOMIMETICS, INC., Gaithersburg, MD (US)

(72) Inventors: John L. Magnani, Gaithersburg, MD (US); Arun K. Sarkar, North Potomac, MD (US); John M. Peterson, Slate Hill, NY (US)

(73) Assignee: GLYCOMIMETICS, INC., Gaithersburg, MD (US)

(21) Appl. No.: 14/439,462

(22) PCT Filed: Oct. 31, 2013
(86) PCT No.: PCT/US2013/067711

§ 371 Date: Apr. 29, 2015

Related U.S. Application Data
(60) Provisional application No. 61/720,627, filed on Oct. 31, 2012.

(51) Int. Cl. C07H 15/24; A61N 5/10; C07H 15/203

(52) U.S. Cl. CPC C07H 15/24; C07H 15/203; A61N 5/10

(57) ABSTRACT

Provided herein are E-selectin antagonist therapeutic agents and improvements thereto and compositions comprising these E-selectin antagonists. Methods are also provided for using these E-selectin antagonist therapeutic agents to treat and/or prevent diseases and disorders treatable by inhibiting binding of an E-selectin to an E-selectin ligand. Also provided herein improvements to E-selectin antagonist glycomimetic compounds that improve the oral bioavailability of the glycomimetic compounds.

### BACKGROUND

1. Technical Field

E-selectin antagonist therapeutic agents and improvements thereto and compositions comprising these E-selectin antagonists are described herein that may be used for treating diseases and disorders treatable by inhibiting binding of an E-selectin to an E-selectin ligand. Improvements to E-selectin antagonist glycomimetic compounds include modifications that improve the oral bioavailability of the glycomimetic compounds.

2. Description of the Related Art

Selectins include three cell adhesion molecules that have well-characterized roles in leukocyte homing. E-selectin (endothelial selectin) and P-selectin (platelet selectin) are expressed by endothelial cells at sites of inflammation or injury. When leukocytes expressing selectin ligands on their cell surface bind to the respective selection, the leukocytes roll on the activated vasculature (see, e.g., Kansas, Blood 88:3259-87 (1996)). When abnormal adhesion of selectin-mediated cell adhesion occurs tissue damage may result instead of repair. Many pathological conditions such as autoimmune and inflammatory diseases, shock, and reperfusion injuries involve abnormal adhesion of white blood cells. Additional and improved therapeutics are needed to treat or prevent such conditions.

Recent investigations have suggested that cancer cells are immunostimulatory and interact with selectins to extravasate and metastasize (see, e.g., Gout et al., Clin. Exp. Metastasis 25:335-344 (2008); Kannagi et al., Cancer Sci. 95:377-84 (2004); Witz, Immunol. Lett. 104:89-93 (2006); Brodt et al., Int. J. Cancer 71:612-19 (1997)). A number of cancers are highly treatable when treated before the cancer has moved beyond the primary site. However, often once the cancer has spread beyond the primary site, the treatment options are limited and the survival statistics decline dramatically. For example, when colorectal cancer is detected at a local stage (i.e., confined to the colon or rectum), over 90% of those diagnosed survive more than five years. Conversely, when colorectal cancer has spread to distant sites (i.e., metastasized from the primary site to distant sites), the five-year survival rate of those diagnosed drops dramatically to only 11%.

A need exists for improved therapeutics for treating inflammatory diseases and cancers and for reducing the likelihood of metastasis of cancers. A need also exists for enhancing the oral availability of therapeutic agents. The present disclosure fulfills these needs and further provides other related advantages.

### BRIEF SUMMARY

Briefly, provided herein are E-selectin antagonist therapeutic agents, including glycomimetic compounds, that are useful for treating and/or preventing (i.e., reducing the likelihood of occurrence) of a disease, disorder or condition that is treatable by inhibiting binding of E-selectin to one or more E-selectin ligands. Embodiments provided herein include the following.

In one embodiment, the present invention is direct to compounds having the following structure (I):

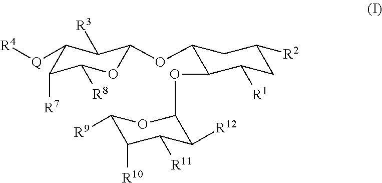

or a pharmaceutically acceptable salt, stereoisomer, tautomer, prodrug or solvate thereof, wherein R¹, R², R³, R⁴, R⁷, R⁸, R⁹, R¹⁰, R¹¹, R¹² and Q are as defined herein. Various other embodiments provide pharmaceutical compositions comprising a compound of structure (I) and a pharmaceutically acceptable carrier, diluent or excipient.

In other embodiments the present disclosure is directed to a composition comprising a compound of formula (Ik), a compound of formula (IId) and a pharmaceutically acceptable carrier, diluent or excipient, wherein the compound of formula (Ik) has the following structure:

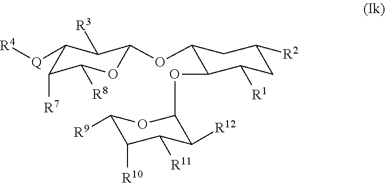

or a pharmaceutically acceptable salt, stereoisomer, tautomer, prodrug or solvate thereof, wherein R¹, R², R³, R⁴, R⁷, R⁸, R⁹, R¹⁰, R¹¹, R¹² and Q are as defined herein, and the compound of formula (IId) has the following structure:

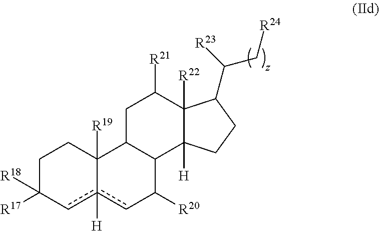

or a pharmaceutically acceptable salt, stereoisomer, tautomer, prodrug or solvate thereof, wherein R¹⁷, R¹⁸, R¹⁹, R²⁰, R²¹, R²², R, R²⁴ and Z are as defined herein.

Other embodiments are directed to a method for decreasing the likelihood of occurrence of metastasis of cancer cells in a subject in need thereof, the method comprises administering to the subject any of the foregoing pharmaceutical compositions.

In another embodiment, a method for decreasing the likelihood of occurrence of infiltration of cancer cells into bone marrow in a subject in need thereof is provided, the method comprises administering to the subject any one of the above described pharmaceutical compositions.

Still other embodiments provide a method for inhibiting adhesion of a tumor cell that expresses a ligand of E-selectin to an endothelial cell expressing E-selectin, the method comprising contacting the endothelial cell with a pharmaceutical composition comprising (a) a pharmaceutically acceptable excipient and (b) a compound of structure (I), permitting the compound to interact with E-selectin present on the endothelial cell, thereby inhibiting binding of the tumor cell to the endothelial cell. For example, in some embodiments the endothelial cell is present in the bone marrow.

Other embodiments are directed to a method for treating a cancer in a subject, the method comprising administering to the subject (a) the compound of structure (I) or any of the above described pharmaceutical compositions and (b) at least one of (i) chemotherapy and (ii) radiotherapy.

In still other embodiments, the present disclosure provides a method for decreasing the likelihood of occurrence of thrombus formation in a subject, comprising administering to the subject the compound of structure (I) or any of the foregoing pharmaceutical compositions.

In yet more embodiments, a method for enhancing hematopoietic stem cell survival in a subject is provided, the method comprising administering to the subject the compound of formula (I) or any of the above pharmaceutical compositions. For example, in some embodiments the subject has received or will receive chemotherapy or radiotherapy or both chemotherapy and radiotherapy. In other embodiments, the subject has received or will receive two or more cycles of chemotherapy or radiotherapy.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” In addition, the term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features. Headings provided herein are for convenience only and do not interpret the scope or meaning of the claimed embodiments.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise. Thus, for example, reference to “a compound” may refer to one or more compounds, or a plurality of such compounds, and reference to “a cell” or “the cell” includes reference to one or more cells and equivalents thereof (e.g., plurality of cells) known to those skilled in the art, and so forth. Similarly, reference to “a composition” includes a plurality of such compositions, and refers to one or more compositions unless the context clearly dictates otherwise. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. It should also be noted that the term “or” is generally employed in its sense including “and/or” unless the content clearly dictates otherwise. The term, “at least one,” for example, when referring to at least one compound or to at least one composition, has the same meaning and understanding as the term, “one or more.”

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1 depicts an exemplary method for preparation of compounds of structure (I) having various hydrophobic groups at the R⁵ position.

FIG. 2 is a schematic showing a method for preparing exemplary compounds of structure (I) having hydrophobic moieties at the R² position.

### DETAILED DESCRIPTION

Provided herein are E-selectin antagonist therapeutic agents, including glycomimetic compounds, that are useful for treating and/or preventing (i.e., reducing the likelihood of occurrence) of a disease, disorder or condition that is treatable by inhibiting binding of E-selectin to one or more E-selectin ligands. The glycomimetic compounds described herein are potent E-selectin antagonists. The term “glycomimetic” refers to any naturally occurring or non-naturally occurring carbohydrate compound in which one or more substituents has been replaced, or one or more rings has been modified (e.g., substitution of carbon for a ring oxygen), to yield a compound that is not fully carbohydrate. Moreover, also provided are glycomimetic compounds and compositions comprising glycomimetic compounds that have structural characteristics that render the compounds and compositions orally bioavailable. Accordingly, the E-selectin antagonists provided herein exhibit properties with improved therapeutic efficacy.

In one embodiment provided herein, the E-selectin antagonist is a glycomimetic compound that has the following formula (I):

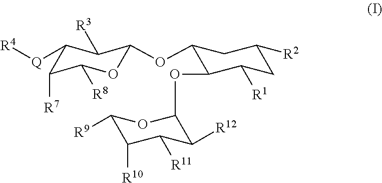

or a pharmaceutically acceptable salt, stereoisomer, tautomer, prodrug or solvate thereof, wherein:

Q is —O—, —S— or —CH₂—;

R¹ is C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₃-C₆ cycloalkyl, or C₃-C₆ halocycloalkyl;

R² is H, -L¹-C₁-C₂₅ alkyl, -L¹-C₂-C₂₅ alkenyl, -L¹-C₂-C₂₅ alkynyl, -L¹-C₁-C₂₅ haloalkyl, -L¹-C₂-C₂₅ haloalkenyl, -L¹-C₂-C₂₅ haloalkynyl or -L¹-M;

R³ is —OC(═O)aryl, —NHC(═O)R³ or -L¹-M;

R⁴ is aryl, aralkyl or has the following structure:

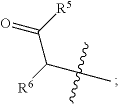

R⁵ is —OR¹⁴, —NHOR¹⁵, or —N(R¹⁵)(R¹⁶);

R⁶ is C₁-C₈ haloalkyl, C₂-C₈ haloalkenyl, C₂-C₈ haloalkynyl, cycloalkylalkyl or halocycloalkylalkyl;

R⁷, R¹⁰, R¹¹ and R¹² are each independently —OH, halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R⁸ is —CH₂OH, C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R⁹ and R¹³ are each independently C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R¹⁴ is H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl;

R¹⁵ and R¹⁶ are each independently H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl;

L¹ is an optional linker; and M is a non-glycomimetic moiety, wherein, the compound comprises at least one of the following:
Q is —S— or —CH₂—;
R¹ is C₂-C₈ alkenyl, C₂-C₈ alkynyl, C₁-C₈ haloalkyl, C₂-C₈ haloalkenyl, C₂-C₈ haloalkynyl, or C₃-C₆ halocycloalkyl;
R² is -L¹-C₂-C₂₅ alkenyl, -L¹-C₂-C₂₅ alkynyl, -L¹-C₁-C₂₅ haloalkyl, -L¹-C₂-C₂₅ haloalkenyl, -L¹-C₂-C₂₅ haloalkynyl or -L¹-M;
R³ is —NHC(═O)R³ or -L¹-M;
R₄ is aryl or aralkyl;
R₄ has the following structure:

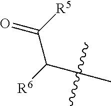

wherein R⁵ is —OR¹⁴ or —N(R¹⁵)(R¹⁶), wherein R¹⁴ is C₃-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl; or R⁶ is C₁-C₈ haloalkyl, C₂-C₈ haloalkenyl, C₂-C₈ haloalkynyl, cyclopropylalkyl, cyclobutylalkyl, cyclopentylalkyl or halocycloalkylalkyl;

at least one of R⁷, R¹⁰, R¹¹ or R¹² is independently halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R⁸ is C₁-C₈ alkyl, C₂-C₈ alkenyl, C₂-C₈ alkynyl, C₁-C₈ haloalkyl, C₂-C₈ haloalkenyl or C₂-C₈ haloalkynyl; or

R⁹ is C₂-C₈ alkenyl, C₂-C₈ alkynyl, C₁-C₈ haloalkyl, C₂-C₈ haloalkenyl or C₂-C₈ haloalkynyl.

In certain embodiments, the compound has the following structure (Ia):

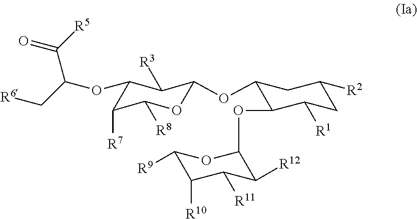

wherein R^{6′} is C₁-C₇ haloalkyl, C₂-C₇ haloalkenyl, C₂-C₇ haloalkynyl, cycloalkyl or halocycloalkyl.

In other embodiments, at least one of R⁷, R¹⁰, R¹¹ or R¹² is —OH.

In some other embodiments, R⁸ is —CH₂OH.

In some more embodiments, R⁹ is methyl.

In still other embodiments, R¹ is methyl or ethyl.

In some other exemplary embodiments, the compound has the following structure (Ib):

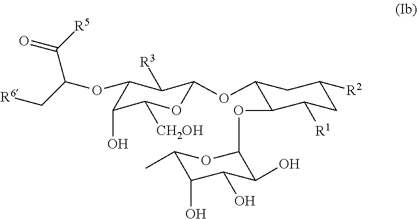

In some of the foregoing embodiments, R^{6′} is C₃-C₆ cycloalkyl. For example, in some embodiments R^{6′} is cyclopropyl or cyclohexyl, and the compound has one of the following structures (Ic) or (Id).

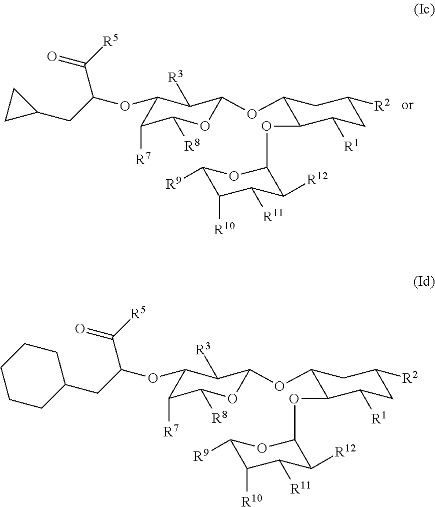

In some other embodiments, R⁶ is C₁-C₈ haloalkyl, C₂-C₈ haloalkenyl or C₂-C₈ haloalkynyl. For example, in some embodiments R⁶ is C₁-C₈ fluoroalkyl, C₂-C₈ fluoroalkenyl or C₂-C₈ fluoroalkynyl.

In still other embodiments, the compound has one of the following structures (le), (If) or (Ig):

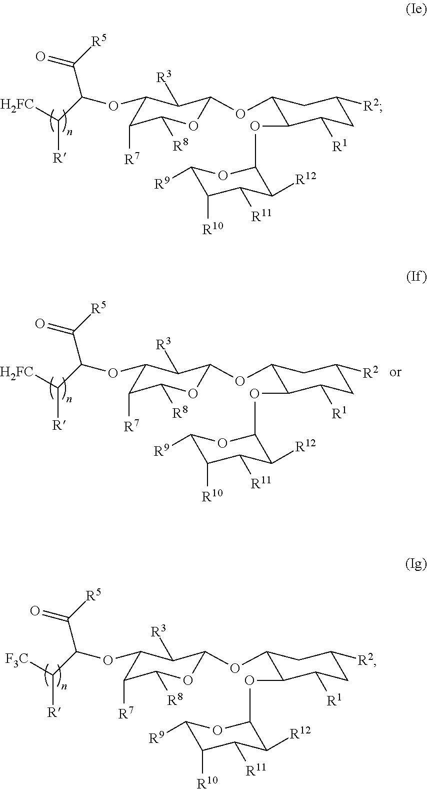

wherein:

R′ is, at each occurrence, independently H, halo, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl or C₃-C₆ halocycloalkyl group;

n is an integer from 1 to 7,

wherein R′ and n are selected such that R⁶ comprises no more than eight acyclic carbon atoms.

In even more embodiments, R⁵ is —OH, —NHOCH₃, —NHOH, —N(CH₃)₂ or —NH(CHF₂).

In some other exemplary embodiments, the compound has the following structure (Ih):

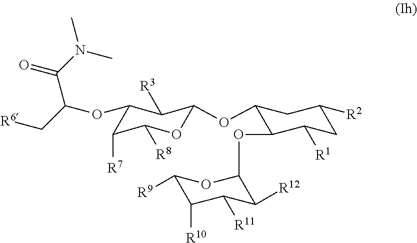

wherein R^{6′} is C₁-C₇ haloalkyl, C₂-C₇ haloalkenyl, C₂-C₇ haloalkynyl, C₃-C₆ cycloalkyl or C₃-C₆ halocycloalkyl.

In some other embodiments, R⁵ is —OR¹³ and R¹³ is C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl. For example, in some embodiments the compound has the following structure (Ii):

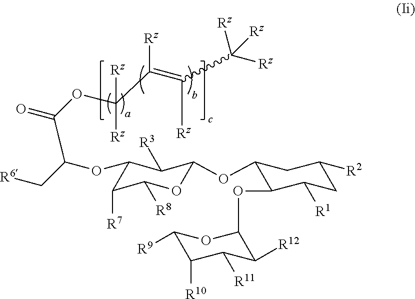

wherein:

R^{6′} is C₁-C₇ haloalkyl, C₂-C₇ haloalkenyl, C₂-C₇ haloalkynyl, C₃-C₆ cycloalkyl or C₃-C₆ halocycloalkyl;

R^{z} is, at each occurrence, independently H or halogen;

a and b are, at each occurrence, independently 0 or 1; and

c is an integer from 5 to 24,

wherein a, b and c are selected such that R¹³ comprises from 6 to 25 carbon atoms.

In other embodiments of the foregoing, the compound has one of the following structures:

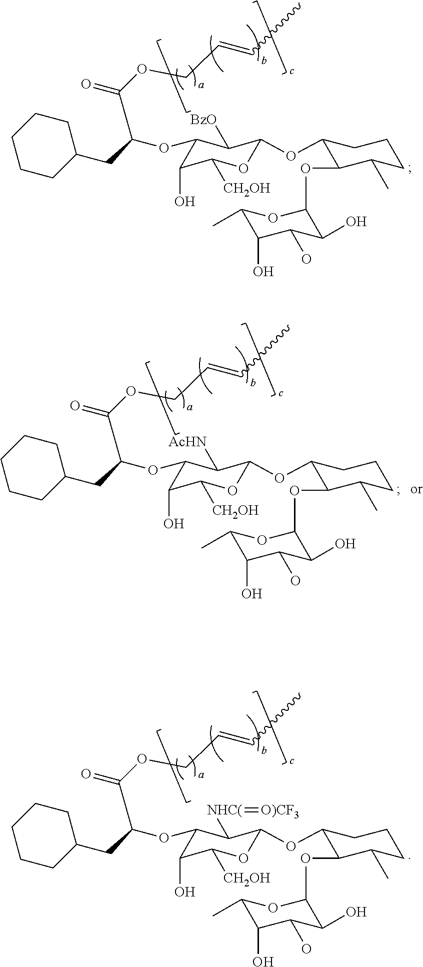

In even more embodiments, R² is -L¹-C₁-C₂₅ alkyl, -L-C₂-C₂₅ alkenyl, -L¹-C₂-C₂₅ alkynyl, -L¹-C₁-C₂₅ haloalkyl, -L¹-C₂-C₂₅ haloalkenyl or -L¹-C₂-C₂₅ haloalkynyl. For example, in some embodiments the compound has the following structure (Ij):

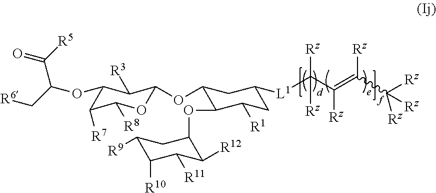

wherein:

R^{6′} is C₁-C₇ haloalkyl, C₂-C₇ haloalkenyl, C₂-C₇ haloalkynyl, C₃-C₆ cycloalkyl or C₃-C₆ halocycloalkyl;

R^{z} is, at each occurrence, independently H or halogen;

d and e are, at each occurrence, independently 0 or 1; and

f is an integer from 5 to 24,

wherein d, e and f are selected such that the alkyl or alkenyl moiety in R² comprises from 6 to 25 carbon atoms.

In other embodiments, the compound has the following structure:

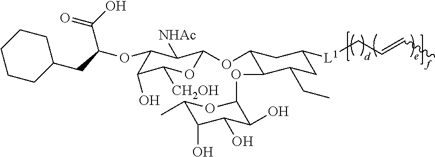

In other embodiments, R² is H.

In some other embodiments, R² is -L¹-M. In still other embodiments, R³ is -L¹-M. For example, in some embodiments M is a steroidal moiety. In even more embodiments, the steroidal moiety is cholic acid or a derivative thereof.

In some other exemplary embodiments, M has the following structure (II):

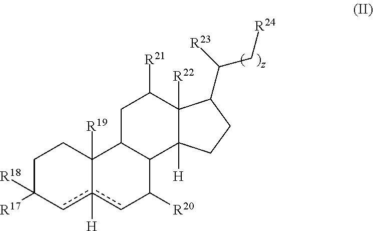

or a pharmaceutically acceptable salt, stereoisomer, tautomer, prodrug or solvate thereof, wherein:

R¹⁷ is H, —OH, —N₃, R²⁶, —N(R²⁷)(R²⁸), —NHC(═O)R²⁷, —C(═O)N(R²⁷)(R²⁸), —OC(═O)Ar, —NC(═O)Ar, —OC(═O)OR²⁹ or —OC(═O)R²⁹ or R¹⁷ joins with R¹⁸ to form oxo or ═NCH₂Ar;

R¹⁸ is H or —NH₂ or R¹⁸ joins with R¹⁷ to form oxo or ═NCH₂Ar;

R¹⁹, R²² and R²³ are each independently H, C₁-C₈ alkyl;

R²⁰ and R²¹ are each independently H, —OH, or R²⁶;

R²⁴ is R²⁶, —C(═O)OR³⁰, —CH₂OR²⁹, —C(═O)N(R³¹)(R³²), —C(═O)SR³⁰, —CH₂S(O)ₚ—SR³⁰, —CH₂N(R²⁷)(R²⁸) or —CH₂S(O)ₚ—SR³⁰;

R²⁶ is a direct bond to L¹ or a direct bond to a compound of structure (I);

R²⁷ and R²⁸ are each independently H, C₁-C₄ alkyl, C₃-C₇ cycloalkyl, C₄-C₁₀ alkylcycloalkyl or aryl or R²⁷ joins with R²⁸ to form a 4, 5, 6 or 7-membered heterocycle;

R²⁹ is H or C₁-C₃ alkyl;

R³⁰ is H C₁-C₁₀ alkyl, C₂-C₁₀ alkenyl, C₂-C₁₀ alkynyl, aryl or aralkyl;

R³¹ and R³² are each independently H, C₁-C₄ alkyl, C₃-C₇ cycloalkyl, C₄-C₁₀ alkylcycloalkyl, polyalkylamine or aryl or R³¹ joins with R³² to form a mono, bi or tricyclic heterocycle containing from 1 to 5 nitrogen atom;

Ar is optionally substituted phenyl;
p and z are each independently 0, 1 or 2; and

a dashed line indicates an optional double bond,

wherein all valences are satisfied.

In some embodiments of the foregoing, the compound of structure (II) has one of the following structures (II′) or (II″):

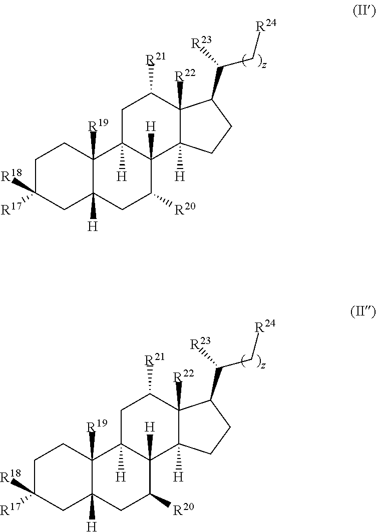

In still other embodiments, R¹⁹, R²² and R²³ are each methyl.

In other embodiments, R¹⁸ is H, and in even more other embodiments R¹⁸ is —NH₂.

In some embodiments, R¹⁷ is —OH.

In still other embodiments, at least one of R²⁰ or R²¹ is H. For example, in some embodiments R²¹ is H.

In even more embodiments, at least one of R²⁰ or R²¹ is —OH. For example, in some embodiments each of R²⁰ and R²¹ is —OH.

In other embodiments, R²⁴ is —CO₂CH₃.

In some embodiments of the foregoing, at least one of R¹⁷, R¹⁸, R²¹ or R²⁴ is R²⁶. For example, in some embodiments at least two of R¹⁷, R²⁰, R²¹ or R²⁴ are R²⁶, such that the compound of structure (II) comprises two compounds of structure (I) covalently bound thereto.

In some embodiments, L¹ is present. For example, in some embodiments L¹ comprises methylene, ester, amide or ether functional groups or combinations thereof. In other embodiments, L¹ has one of the following structures:

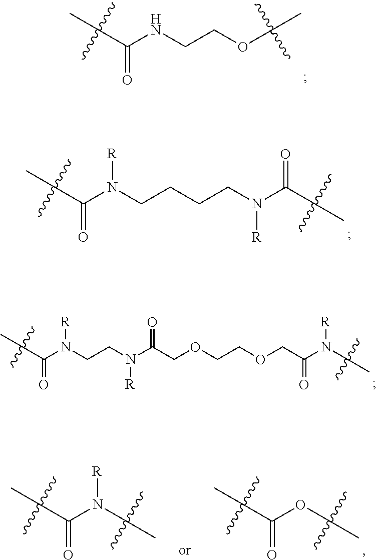

wherein each R is independently H or C₁-C₆ alkyl.

In still other embodiments, R³ is —NHC(═O)CH₃, —NHC(═O)CH₃, —OC(═O)phenyl or —OC(═O)cyclopropyl.

In some other exemplary embodiments, R⁸ is C₁-C₈ haloalkyl. For example, in some embodiments R⁸ is —CH₂CHX₂. In even more embodiments of the foregoing, X is F.

In some other embodiments, R² is -L¹-C₁-C₈ haloalkyl or at least one of R¹, R⁷, R⁸, R⁹, R¹², R¹³, R¹⁴, R¹⁵ or R¹⁶ is C₁-C₈ haloalkyl. For example, in some embodiments at least two of R¹, R⁷, R⁸, R⁹, R¹², R¹³, R¹⁴, R¹⁵ or R¹⁶ are C₁-C₈ haloalkyl. In even more embodiments, at least one C₁-C₈ haloalkyl is —CH₂(CH₂)ₘ—X, —(CH₂)ₘ—CHX₂, or —(CH₂)ₘ—CX₃, wherein m is an integer from 0 to 6 and X is F, Cl, Br or I. For example, in some embodiments X is F.

In some embodiments, R¹ is C₁-C₈ haloalkyl. For example, in some embodiments R¹ is —CH₂CHX₂. In some other exemplary embodiments, X is F.

In some more embodiments, R⁴ is aryl or aralkyl. For example, in some embodiments R⁴ has one of the following structures:

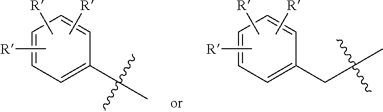

wherein each R′ is independently halo or hydroxyl.

In still other embodiments, R⁴ has one of the following structures:

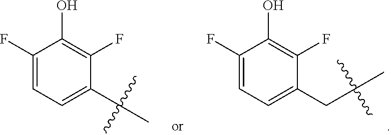 [SMILES: *c1ccc(F)c(O)c1F ; *Cc1ccc(F)c(O)c1F]

In some embodiments of the foregoing, Q is —O—. In other embodiments Q is —S—. In still other embodiments, Q is —CH₂—.

In certain embodiments, the compound has one of the following structures:

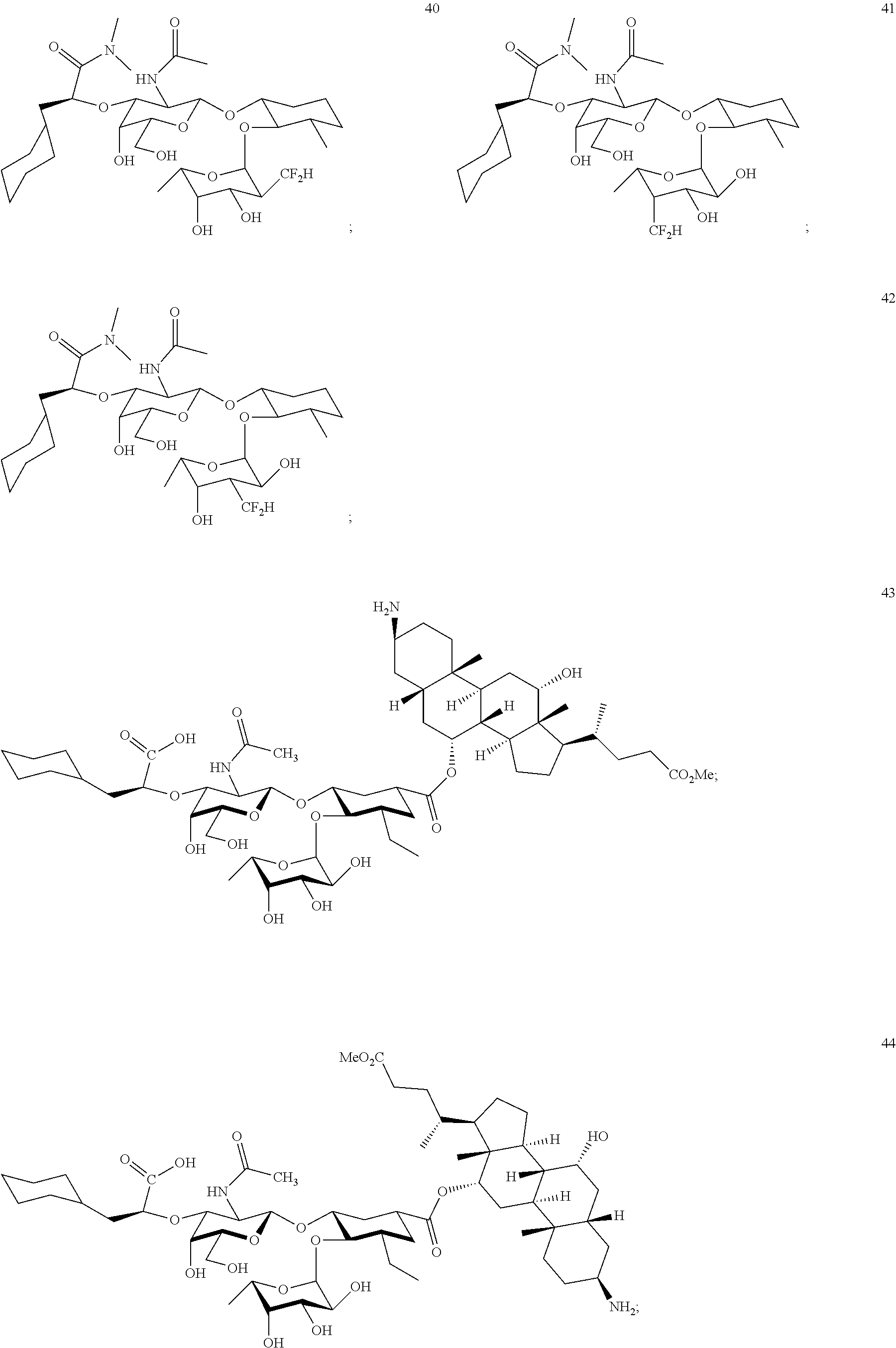

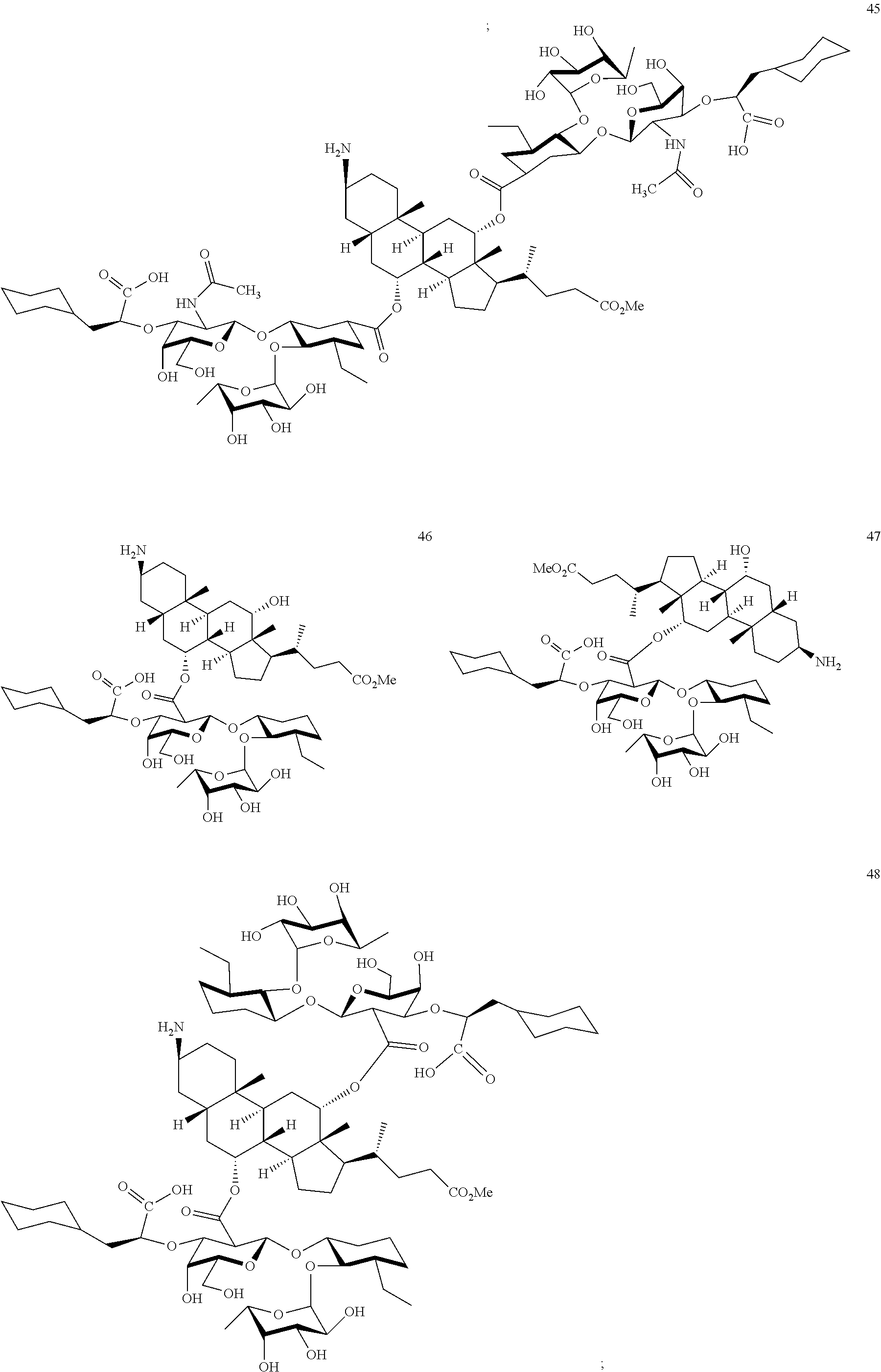

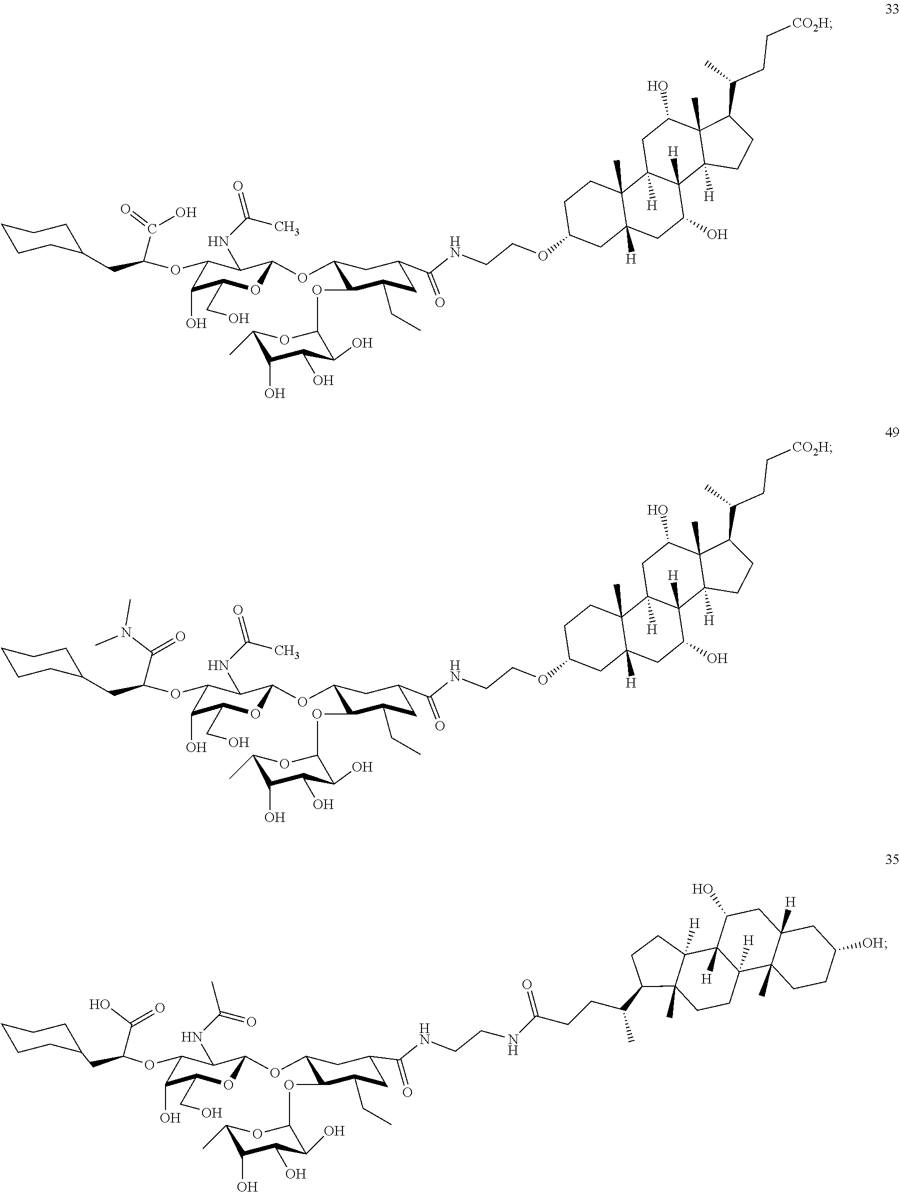

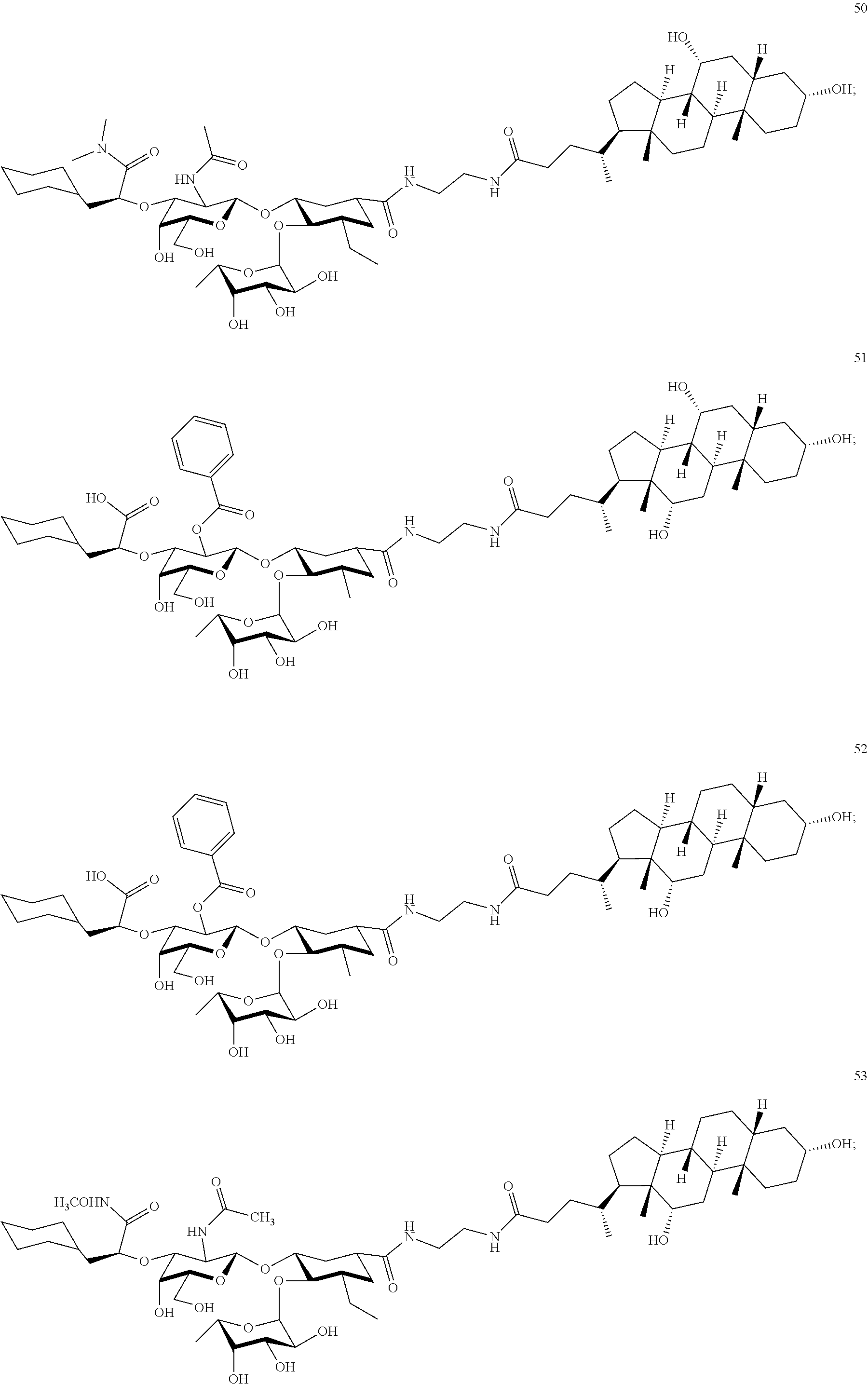

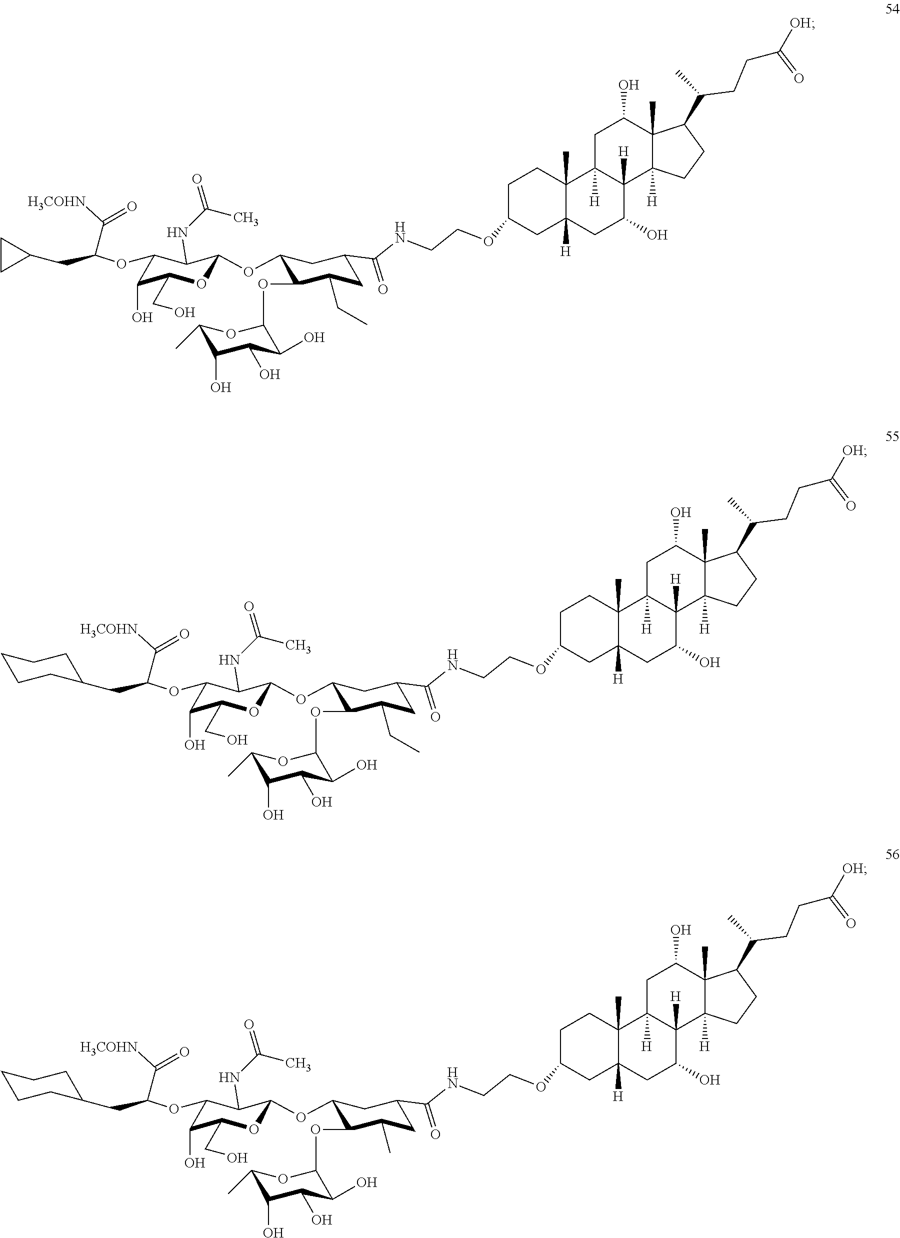

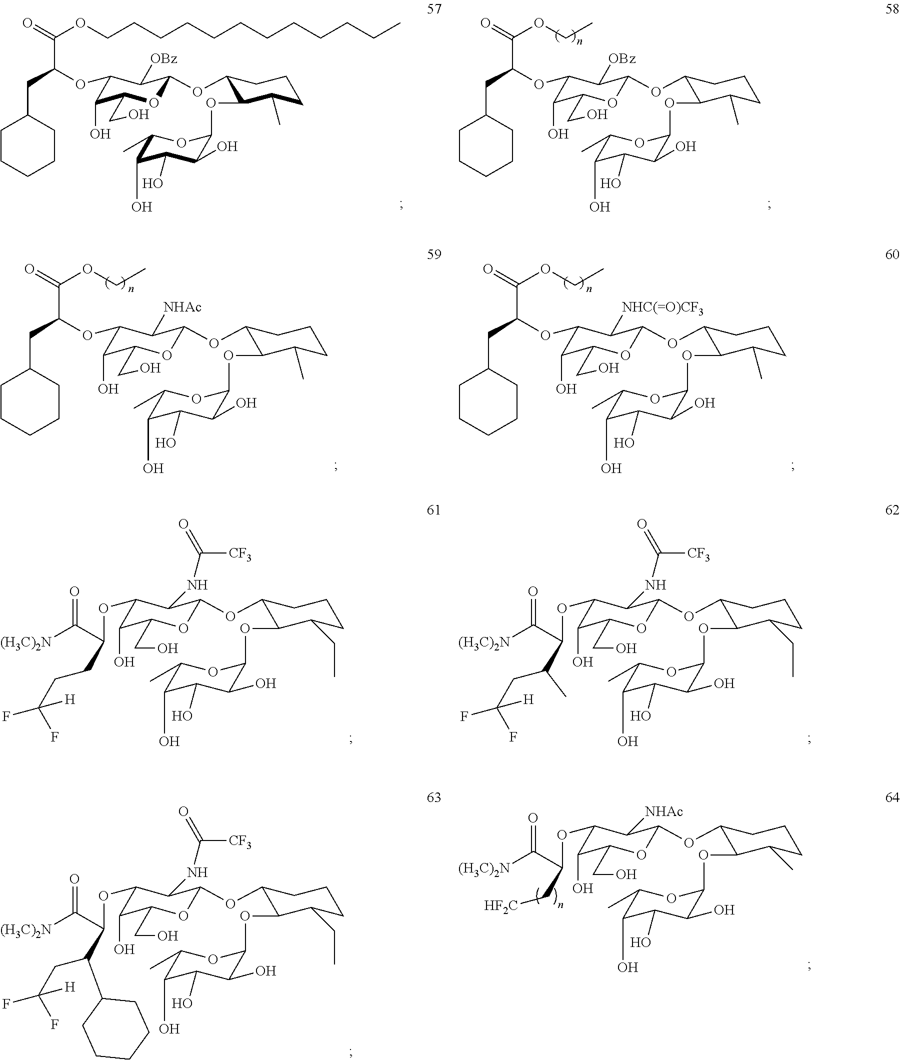

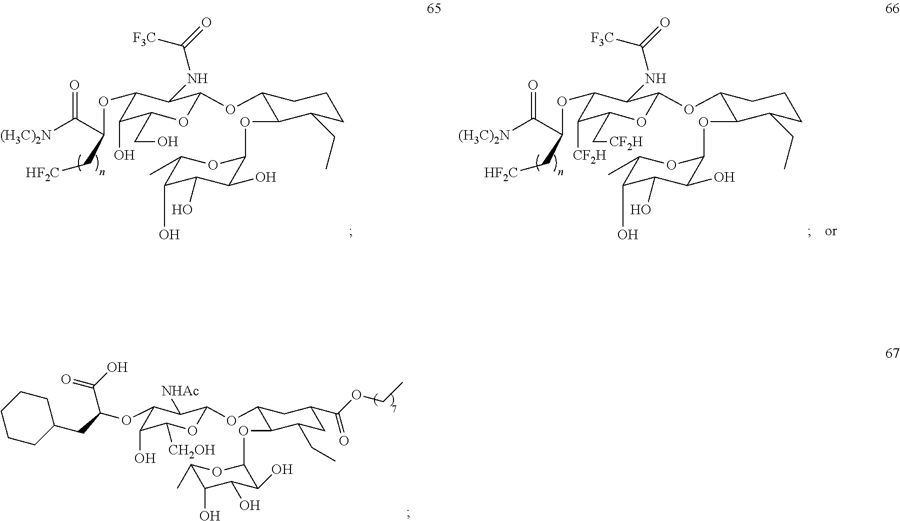

wherein n is an integer from 1-8.

It is understood that in the present description, combinations of substituents and/or variables of the depicted formulae are permissible only if such contributions result in stable compounds.

Also provided herein are pharmaceutical compositions that comprise one or more of the compounds of formula (I), substructures and specific structures thereof, and a pharmaceutically acceptable excipient. A compound of formula (I) or a pharmaceutical composition comprising the compound may be used in methods described herein for decreasing the likelihood of occurrence of metastasis of cancer cells (also called tumor cells herein) in a subject (i.e., individual, patient) who is in need thereof by administering the compound or composition to the subject. In other embodiments, a compound of formula (I) or a pharmaceutical composition comprising the compound may be used in methods for decreasing the likelihood of occurrence of infiltration of cancer cells into bone marrow in a subject who is in need thereof by administering the compound or composition to the subject. In still another embodiment, methods are provided herein for inhibiting adhesion of a cancer cell that expresses a ligand of E-selectin to an endothelial cell expressing E-selectin on the cell surface of the endothelial cell wherein the method comprises contacting the endothelial cell and the compound or composition comprising the compound (i.e., in some manner permitting the compound or composition comprising the compound to interact with the endothelial cell) such that the compound interacts with E-selectin on the endothelial cell, thereby inhibiting binding of the cancer cell to the endothelial cell. In certain embodiments, the endothelial cell is present in the bone marrow. In still another embodiment described herein, a method is providing for treating a cancer in a subject in need thereof by administering a compound of formula I or a composition comprising the compound to the subject. The compound (or composition comprising the compound) may be administered in conjunction with (i.e., as an adjunct therapy, which is also called adjunctive therapy) with chemotherapy or radiation or both.

The chemotherapy or radiation therapy or combination thereof may be referred to as the primary anti-tumor or anti-cancer therapy that is being administered to the subject to treat the particular cancer. In another embodiment, a method is provided herein for reducing (i.e., inhibiting, diminishing) chemosensitivity or radiosensitivity of hematopoietic stem cells (HSC) to the chemotherapeutic drug(s) or radioactive therapy, respectively, in a subject in need thereof, comprising administering to the subject one or more of the E-selectin antagonist glycomimetic compounds described herein. In another embodiment, methods are provided for enhancing (i.e., promoting) survival of hematopoietic stem cells in a subject in need thereof, comprising administering one or more of the E-selectin antagonist compounds described herein. The glycomimetic compounds may be used for treating any one or more of the diseases or conditions described herein or for the preparation or manufacture of a medicament for use in treating any one or more of the diseases or conditions described herein. Each of these methods and uses are described in greater detail.

### DEFINITIONS

The terms below, as used herein, have the following meanings, unless indicated otherwise. Certain chemical groups named herein are preceded by a shorthand notation indicating the total number of carbon atoms that are to be found in the indicated chemical group.

As used herein, the following terms have the following meanings.

“Amino” refers to the —NH₂ radical.

“Cyano” refers to the —CN radical.

“Halo” refers to the fluoro, chloro, bromo or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Alkyl” refers to a straight or branched hydrocarbon chain radical consisting solely of carbon and hydrogen atoms, which is saturated, having from one to twenty-five carbon atoms (C₁-C₂₅ alkyl), one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond. Exemplary alkyl groups include, but are not limited to, methyl, ethyl, n-propyl, 1-methylethyl(iso-propyl), n-butyl, n-pentyl, 1,1-dimethylethyl(t-butyl), 3-methylhexyl, 2-methylhexyl, and the like. Unless stated otherwise specifically in the specification, an alkyl group may be optionally substituted.

“Alkenyl” refers to a straight or branched hydrocarbon chain radical consisting solely of carbon and hydrogen atoms, which comprises at least one carbon-carbon double bond. Alkenyls may comprise carbon-carbon single bonds and/or carbon-carbon triple bonds, provided at least one carbon-carbon double bond is present. Alkenyls have from two to twenty-five carbon atoms (C₂-C₂₅ alkenyl), two to twelve carbon atoms (C₂-C₁₂ alkenyl), two to eight carbon atoms (C₂-C₈ alkenyl) or two to six carbon atoms (C₂-C₆ alkenyl), and which is attached to the rest of the molecule by a single bond. Exemplary alkenyl groups include, but are not limited to ethenyl, prop-1-enyl, but-1-enyl, pent-1-enyl, penta-1,4-dienyl, hexenyl and the like. Unless stated otherwise specifically in the specification, an alkenyl group may be optionally substituted.

“Alkynyl” refers to a straight or branched hydrocarbon chain radical consisting solely of carbon and hydrogen atoms, which comprises at least one carbon-carbon triple bond. Alkynyls may comprise carbon-carbon single bonds and/or carbon-carbon double bonds, provided at least one carbon-carbon triple bond is present. Alkynyls have from two to twenty-five carbon atoms (C₂-C₂₅ alkynyl), two to twelve carbon atoms (C₂-C₁₂ alkynyl), two to eight carbon atoms (C₂-C₈ alkynyl) or two to six carbon atoms (C₂-C₆ alkynyl), and which is attached to the rest of the molecule by a single bond. Exemplary alkynyl groups include, but are not limited to ethynyl, propynyl, butynyl, pentynyl, hexynyl, and the like. Unless stated otherwise specifically in the specification, an alkynyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group may be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group may be optionally substituted.

“Thioalkyl” refers to a radical of the formula —SRₐ where Rₐ is an alkyl, alkenyl or alkynyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of certain embodiments of the disclosed compounds, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms (C₃-C₁₅), three to ten carbon atoms (C₃-C₁₀), three to 6 carbon atoms (C₃-C₆) or three to 5 carbon atoms (C₃-C₅), and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Alkylcycloalkyl” refers to a radical of the formula —R_{b}R_{d} where R_{b} is a cycloalkyl moiety as defined above and R_{d} is an alkyl radical as defined above. Unless stated otherwise specifically in the specification, a alkylcycloalky group may be optionally substituted.

“Cycloalkylalkyl” refers to a radical of the formula —R_{b}R_{d} where R_{b} is an alkylene chain as defined above and R_{d} is a cycloalkyl radical as defined above. Unless stated otherwise specifically in the specification, a cycloalkylalkyl group may be optionally substituted.

“Cycloalkylalkoxy” refers to a radical of the formula —ORₐ where Rₐ is a cycloalkyl group as defined above. Unless stated otherwise specifically in the specification, a cycloalkylalkoxy group may be optionally substituted.

“Fused” refers to any ring structure described herein which is fused to an existing ring structure in certain embodiments of the disclosed compounds. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A “fluoroalkyl” is a haloalkyl, which comprises at least one fluorine substitution. Unless stated otherwise specifically in the specification, a haloalkyl group may be optionally substituted.

“Haloalkenyl” refers to an alkenyl radical, as defined above, that is substituted by one or more halo radicals, as defined above, e.g., fluoroethenyl, 1,2-difluoroethenyl, 3-bromo-2-fluoropropenyl, 1,2-dibromoethenyl, and the like. A “fluoroalkenyl” is a haloalkenyl, which comprises at least one fluorine substitution. Unless stated otherwise specifically in the specification, a haloalkenyl group may be optionally substituted.

“Haloalkynyl” refers to an alkynyl radical, as defined above, that is substituted by one or more halo radicals, as defined above, e.g., fluoroethynyl, 1,2-difluoroethynyl, 3-bromo-2-fluoropropynyl, 1,2-dibromoethynyl, and the like. A “fluoroalkynyl” is a haloalkynyl, which comprises at least one fluorine substitution. Unless stated otherwise specifically in the specification, a haloalkynyl group may be optionally substituted.

“Halocycloalkyl” refers to a cycloalkyl radical as defined above, that is substituted by one or more halo radicals. Non limiting examples of halocycloalky groups include fluorocyclopropane, fluorocyclohexane and the like. Unless stated otherwise specifically in the specification, a halocycloalkyl group may be optionally substituted.

“Halocycloalkylalkyl” refers to a radical of the formula —R_{b}R_{d} where R_{b} is an alkylene chain as defined above and R_{d} is a cycloalkyl radical as defined above and wherein the alkylene chain and/or the cycloalkyl radical comprises one or more halo substitutions. Unless stated otherwise specifically in the specification, a halocycloalkylalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of certain embodiments of the compounds, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Hydroxylalkyl,” “hydroxylalkenyl” and “hydroxylalkynyl” refer to an alkyl, alkenyl or alkynyl radical, respectively, as defined above, that is substituted by one or more hydroxyl groups. The hydroxyl groups may be primary, secondary or tertiary. Unless stated otherwise specifically in the specification, a hydroxylalkyl, hydroxylalkenyl and hydroxylalkynyl group may be optionally substituted.

The term “substituted” used herein means any of the above groups (i.e., alkyl, alkenyl, alkynyl, alkylene, alkoxy, alkylamino, thioalkyl, aryl, aralkyl, cycloalkyl, alkylcycloalkyl, cycloalkylalkyl, cycloalkylalkoxy, haloalkyl, haloalkenyl, haloalkynyl, halocycloalkyl, halocycloalkylalkyl, heterocyclyl, N-heterocyclyl, heterocyclylalkyl, heteroaryl, N-heteroaryl, hydroxylalkyl, hydroxylalkenyl, and/or hydroxylalkynyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R, and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Prodrug” is meant to indicate a compound that may be converted under physiological conditions or by solvolysis to a biologically active compound, for example a compound of structure (I). Thus, the term “prodrug” refers to a metabolic precursor of a compound of certain embodiments that is pharmaceutically acceptable. A prodrug may be inactive when administered to a subject in need thereof, but is converted in vivo to an active compound. Prodrugs are typically rapidly transformed in vivo to yield the parent compound (e.g., compound of structure (I)), for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release an active compound in vivo when such prodrug is administered to a mammalian subject. Prodrugs of a compound of certain embodiments herein may be prepared by modifying functional groups present in the compound in such a way that the modifications are cleaved, either in routine manipulation or in vivo, to the parent compound. Prodrugs include compounds wherein a hydroxy, amino or mercapto group is bonded to any group that, when the prodrug of the compound is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in any of the compounds described herein.

The present disclosure is also meant to encompass all pharmaceutically acceptable compounds of the disclosed compounds (e.g., structure (I) and (II)) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I) or (II), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

The present disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, certain embodiments include compounds produced by a process comprising administering a compound described herein to a mammal for a period of time sufficient to yield a metabolic product thereof. Such products are typically identified by administering a radiolabelled compound in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the disclosed compounds. As used herein, the term “solvate” refers to an aggregate that comprises one or more molecules of any of the disclosed compounds with one or more molecules of solvent. The solvent may be water, in which case the solvate may be a hydrate. Alternatively, the solvent may be an organic solvent. Thus, the presently disclosed compounds may exist as a hydrate, including a monohydrate, dihydrate, hemihydrate, sesquihydrate, trihydrate, tetrahydrate and the like, as well as the corresponding solvated forms. Certain embodiments of the compounds may be true solvates, while in other cases, some embodiments of the compounds may merely retain adventitious water or be a mixture of water plus some adventitious solvent.

Certain embodiments of the compounds, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. Different embodiments of the compounds herein include various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. Some embodiments of the disclosed compounds include tautomers of any said compounds.

A “non-glycomimetic moiety” refers to a moiety having a structure not intended to mimic a carbohydrate molecule. A non-glycomimetic moiety is typically not active as an E selectin antagonist. Instead, non-glycomimetic moieties are generally moieties added to a glycomimetic moiety for purposes of altering the solubility, bio-availability, lipophilicity or other drug-like properties of the glycomimetic. Non-limiting examples of non-glycomimetic moieties include steroidal compounds such as cholic acid, fatty acids, lipids, ampiphilic compounds, and the like.

“Steroid” or “steroidal moiety” refers to a compound or moiety that contains a characteristic arrangement of four cycloalkane rings that are joined to each other. The core of a steroid comprises twenty carbon atoms bonded together that take the form of four fused rings: three cyclohexane rings and one cyclopentane ring. Non-limiting examples of steroidal moieties include cholic acid, cholesterol and derivatives thereof.

### Compound Synthesis Procedures

Synthesis of the compounds of structure I (and substructures and specific structures thereof) may be performed as described herein, including the Examples, using techniques familiar to a person skilled in the art. In general, compounds of structure (I) can be prepared according to General reaction Scheme I below. It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

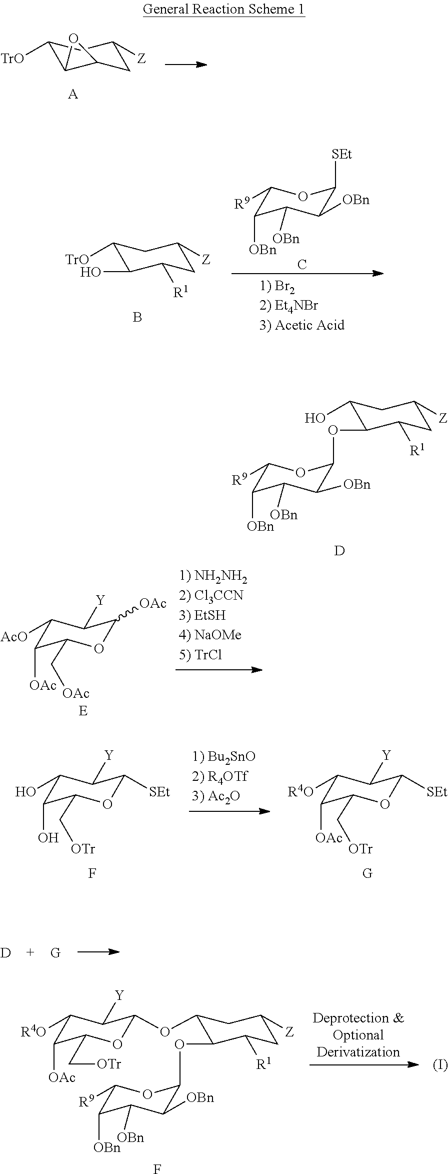

Referring to General Reaction Scheme 1, compounds of structure A, wherein Z is H or acetyl, can be purchased from commercial sources or prepared according to methods known in the art. The epoxide ring of A can be opened using known procedures to yield B. In certain embodiments wherein R₁ is methyl or ethyl, compounds of structure B can be prepared by reaction of A with 1,3-dithiane or 3-methyl-1,3-dithiane and butyl lithium followed by treatment with Raney nickel. Optional fluorination of the thiane intermediate yields compounds having fluorine substitutions at R¹. Reaction of B with C yields then disaccharide D.

In a parallel scheme, compound E, wherein Y is R³ or a protected form thereof, can be purchased or prepared according to known techniques. Compound F is prepared from E under appropriate conditions. Exemplary conditions for such transformation is noted in General reaction Scheme 1 (note that compounds of structure C can be prepared using analogous methods). R⁴ is introduced into the molecule by means of the corresponding triflate derivative and the remaining free alcohol is protected as an acetyl to yield G. D and G are then coupled under appropriate conditions (e.g., in a manner analogous to that described for B+C) to yield F. Compound F is then deprotected to yield various compounds of structure I.

The compounds of structure (I) obtained via the above exemplary scheme can be further derivatized to obtain different compounds of structure (I). For example, lipid moieties (e.g., C₆-C₂₅ alkyl, alkenyl or alkynyls, including halo derivatives thereof) can be added at the R² and or R⁵ position by reaction of an appropriate hydroxyl lipid with an acid (e.g., obtained by saponification of Y, when Y is CO₂Me) and an activating reagent such as DCC or HATU. Other R⁵ moieties can be added using analogous carbonyl chemistries known in the art. Various other modifications to the above General Reaction Scheme I, such as varying the starting material or modifying any of the reaction products to include other non-hydroxyl moieties at R⁷, R⁸, R⁹, R¹⁰, R¹¹ and/or R¹² are possible according methods known in the art.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of the compounds described herein may not possess pharmacological activity as such, they may be administered to a mammal and thereafter metabolized in the body to form embodiments of the described compounds which are pharmacologically active. Such derivatives may therefore be described as “prodrugs”. All prodrugs of the disclosed compounds are included within the scope of the present disclosure.

Furthermore, all compounds herein which exist in free base or acid form can be converted to their pharmaceutically acceptable salts by treatment with the appropriate inorganic or organic base or acid by methods known to one skilled in the art. In some embodiments, salts of the compounds can be converted to their free base or acid form by standard techniques.

### Oral Bioavailability

Orally administered drugs must pass through common sites of metabolism (e.g., the intestinal wall and the portal circulation to the liver) prior to the agent reaching systemic circulation. Hydrophilic drugs are often poorly absorbed when administered orally and are too rapidly eliminated from a subject to exhibit a satisfactory therapeutic effect. Even when absorption of an agent from the subject's gastrointestinal (GI) tract is sufficient, the bioavailability of the agent (or an active metabolite thereof) may be too low to have a sufficient therapeutic effect.

Bioavailability refers to how much of and how fast a therapeutic agent (or active metabolite thereof) enters systemic circulation. Low bioavailability may result one or more factors, including the chemical structure of the therapeutic agent, biological sequelae of the disease to be treated, or the particular subject. Certain therapeutic agents may lack the capability to dissolve readily or to penetrate the epithelial membrane, which may be due, at least in part, to the highly ionized or polar nature of the agent. Therapeutic agents may be susceptible to chemical reactions, such as hydrolysis by gastric acid or digestive enzymes, that reduce absorption and thereby decrease bioavailability. Factors related to the subject to be treated that can affect bioavailability include age, sex, physical activity, genetic phenotype, stress, disorders (e.g., achlorhydria or malabsorption syndromes), and previous GI surgery (e.g., bariatric surgery).

As set forth herein, the oral availability of a glycomimetic may be enhanced by one of several different approaches. Additionally, each approach may be combined in any way, so as to utilize the resultant combination of any two, any three, etc. approaches.

In certain embodiments described herein, oral availability of a glycomimetic is enhanced by increasing its hydrophobicity (i.e., decreasing the polar surface area) and increasing its log D. For example, replacing a hydroxyl group with fluorinated bioisosters, hydrogen or an aliphatic carbon chain may decrease the polar surface area. A general rule used in the art is “Lipinski's Rule” to approximate whether a compound is sufficiently nonpolar to be reasonably likely to be orally active (see, e.g., Lipinski et al., Adv. Drug Deliv. Review 23:3-25 (1997)).

In an embodiment, oral availability of a glycomimetic is enhanced by modifying a compound such that it is actively transported across a cell membrane. An exemplary active transport mechanism is targeting the bile acid active transport system by covalently linking the glycomimetic compounds described herein to a steroid such as cholic acids and derivatives thereof.

In an embodiment, oral availability is achieved by enhancing passive transport of the glycomimetic compounds described herein by forming a facial amphiphile with cholic acid or a derivative thereof. The facial amphiphiles may be the active drug itself or in some embodiments the facial amphiphile may be a prodrug, and the active moiety (i.e., glycomimetic) is attached to cholic acid (or derivative thereof) via a labile linkage which is cleaved in vivo.

In another embodiment, oral availability of a glycomimetic is enhanced by increasing the hydrophobicity of a glycomimetic compound for passive transport by incorporating a lipid-like aliphatic chain to the compound. Such compounds may also be prepared as prodrugs as described above. See also, e.g., Bowe et al., Proc. Natl. Acad Sci. USA 94:12218-23 (1997); Walker et al., Proc. Natl. Acad Sci. USA 93:1585-90 (1996); International Patent Application Publication No. WO 93/11772.

In still another embodiment, compositions are provided that comprise a glycomimetic compound of structure (I) in combination with a cholic acid moiety or derivative thereof (e.g., a glycosylated cholic acid derivative).

In some embodiments, the compounds may be transported via both active and passive transport mechanisms. For example, both mechanisms may operate on a single compound, or in certain embodiments compositions comprising both actively and passively transported compounds are provided. Examples of actively and passively transported compounds include those described in the foregoing paragraph.

### Methods for Characterizing Therapeutic Agents

Characterizing at least one biological activity of a glycomimetic compound or other therapeutic agent described herein may be determined by performing one or more in vitro and in vivo studies routinely practiced in the art and described herein or in the art. In vitro assays include without limitation binding assays, immunoassays, competitive binding assays and cell based activity assays. Non-human primate animal models may be used in pre-clinical studies; however, these animal models are not typically employed in the same routine manner as rodent animal studies designed for assessing the effectiveness or other characteristics of a therapeutic.

An inhibition assay may be used to screen for antagonists of E-selectin. For example, an assay may be performed to characterize the capability of a compound or other agent described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, the method comprises immobilizing E-selectin/Ig chimera onto a matrix (e.g., a multi-well plate, which are typically made from a polymer, such as polystyrene; a test tube, and the like); adding a composition to reduce nonspecific binding (e.g., a composition comprising non-fat dried milk or bovine serum albumin or other blocking buffer routinely used by a person skilled in the art); contacting the immobilized E-selectin with the candidate agent in the presence of sLe^{a} comprising a reporter group under conditions and for a time sufficient to permit sLe^{a} to bind to the immobilized E-selectin; washing the immobilized E-selectin; and detecting the amount of sLe^{a} bound to immobilized E-selectin. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of an agent that is characterized by one or more assays and techniques described herein and in the art may be a biological sample that is obtained from a subject who has been treated with the agent. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In certain embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin antagonists include animal model studies. Exemplary animal models for liquid cancers used in the art include but are not limited to multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 April 1; 23(7): 877-889). Animal models for acute lymphoblastic leukemia (ALL) have been used by persons skilled in the art for more than two decades. Numerous exemplary animal models for solid tumor cancers are routinely used and well known to persons skilled in the art.

Oral availability of the E-selectin antagonist agents described herein may be characterized by methods routinely practiced in the art and described herein. By way of example, absorption systems in rat intestinal absorption animal models are used in the art in which the level of an E-selectin antagonist in the serum can be compared in animals that receive the antagonist either intravenously or intraduodenally. Potential bioavailability of an E-selectin glycomimetic compound that has ionizable groups that exist in solution as a mixture of different ionic forms may be determined by an octanol-water distribution coefficient, which is called log D. This log D value describes the hydrophobicity of a compound at any pH value when the compound. The ionization of those groups, and thus the ratio of the ionic forms, depends on the pH. Software to calculate log D values is commercially available. Also described in the art is log P which describes the hydrophobicity of one form only; therefore, the apparent log P value can be different can vary with pH. Calculating polar surface area (PSA) can also be used to characterize the potential oral availability of a glycomimetic compound described herein.

### Methods for Treating or Preventing Diseases or Disorders

The E-selectin antagonist agents described herein (i.e., compounds of structure (I)) may be useful in methods for preventing (i.e., reducing the likelihood of occurrence or recurrence of) and/or treating a disease or disorder treatable by inhibiting an activity of E-selectin (or inhibiting binding of E-selectin to a ligand, which in turn inhibits a biological activity). Focal adhesion of leukocytes to the endothelial lining of blood vessels is a characteristic step in certain vascular disease processes.

The glycomimetic compounds and other E-selectin antagonist therapeutic agents described herein may be used for treating and/or prevent an inflammatory disease. Inflammation comprises reaction of vascularized living tissue to injury. By way of example, although E-selectin-mediated cell adhesion is important to the body's anti-infective immune response, in other circumstances, E-selectin mediated cell adhesion may be undesirable or excessive, resulting in tissue damage instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases or disorders include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the glycomimetic compounds and other E-selectin antagonist therapeutic agents described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischaemic episodes, prevention of restinosis associated with vascular stenting, and for undesirable angiogenesis, for example, angiogenesis associated with tumor growth.

As understood by a person of ordinary skill in the medical art, the terms, “treat” and “treatment,” refer to medical management of a disease, disorder, or condition of a subject (i.e., patient, individual) (see, e.g., Stedman's Medical Dictionary). In general, an appropriate dose and treatment regimen provide at least one glycomimetic compound or other E-selectin antagonist therapeutic agent described herein in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated with the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can also mean prolonging survival when compared to expected survival if a subject were not receiving treatment. Subjects in need of treatment include those who already have the disease, condition, or disorder as well as subjects prone to have or at risk of developing the disease, condition, or disorder, and those in which the disease, condition, or disorder is to be prevented (i.e., decreasing the likelihood of occurrence of the disease, disorder, or condition).

In particular embodiments of the methods described herein, the subject is a human or non-human animal. A subject in need of the treatments described herein may exhibit symptoms or sequelae of cancer disease, disorder, or condition described herein or may be at risk of developing the disease, disorder, or condition. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of a glycomimetic compound or other E-selectin antagonist therapeutic agent described herein in treating or preventing a disease or disorder or condition described herein can readily be determined by a person of ordinary skill in the medical and clinical arts. Determining and adjusting an appropriate dosing regimen (e.g., adjusting the amount of compound per dose and/or number of doses and frequency of dosing) can also readily be determined by a person of ordinary skill in the medical and clinical arts. One or any combination of diagnostic methods, including physical examination, assessment and monitoring of clinical symptoms, and performance of analytical tests and methods described herein, may be used for monitoring the health status of the subject.

### Methods for Treating or Preventing Binding of Cancer Cells to E-Selectin and for Treating Cancer and Metastasis

As discussed in detail herein, a disease or disorder to be treated or prevented (i.e., reduce the likelihood of occurrence or recurrence) is a cancer and related metastasis and includes cancers that comprise solid tumor(s) and cancers that comprise liquid tumor(s). The agents described herein, including glycomimetics and other E-selectin antagonist therapeutic agents described herein may be useful in methods for preventing (i.e., reducing the likelihood of occurrence or recurrence of) and/or treating cancer. In particular embodiments, the agents may be used preventing (i.e., reducing the likelihood of occurrence or recurrence of) and/or treating metastasis and for inhibiting (slowing, retarding, or preventing) metastasis of cancer cells.

In particular embodiments, the compounds of formula (I), including substructures and specific compounds, and therapeutic agents described herein may be used for decreasing (i.e., reducing) the likelihood of occurrence of metastasis of cancer cells in an individual (i.e., subject, patient) who is in need thereof. The compounds and agents described herein may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, either a cancer that comprises solid tumor(s) or a cancer that comprises a liquid tumor.

Such cancers include, for example, colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, the soft, sponge-like tissue in the center of most bones, and lymph nodes and include leukemia (e.g., AML, ALL, CLL, and CML), lymphoma, and myeloma (e.g., multiple myeloma). Lymphomas include Hodgkin lymphoma, which is marked by the presence of a type of cell called the Reed-Sternberg cell, and non-Hodgkin lymphomas, which includes a large, diverse group of cancers of immune system cells. Non-Hodgkin lymphomas can be further divided into cancers that have an indolent (slow-growing) course and those that have an aggressive (fast-growing) course, and which subtypes respond to treatment differently.

The compounds of formula I and agents described herein (or the pharmaceutical composition comprising the compound or agent) may be administered as an adjunct therapy to chemotherapy or radiotherapy or both, which is being delivered to the subject as primary therapy for treating the cancer. The chemotherapy and radiotherapy that may be administered depend upon several factors including the type of cancer, location of the tumor(s), stage of the cancer, age and gender and general health status of the subject. A person skilled in the medical art can readily determine the appropriate chemotherapy regimen or radiotherapy regimen for the subject in need. The person skilled in the medical art can also determine, with the aid of preclinical and clinical studies, when the compound of formula (I) or agent should be administered to the subject, that is whether the compound or agent is administered prior to, concurrent with, or subsequent to a cycle of the primary chemotherapy or radiation treatment.

Also provided herein is a method for inhibiting adhesion of a tumor cell that expresses a ligand of E-selectin to an endothelial cell expressing E-selectin on its cell surface, which method comprises contacting the endothelial cell with the compound of formula (I) or therapeutic agent as described herein, thereby permitting the compound or agent to interact with E-selectin on the endothelial cell surface and inhibiting binding of the tumor cell to the endothelial cell. Without wishing to be bound by theory, inhibiting adhesion of tumor cells to endothelial cells may reduce in a significant manner, the capability of the tumor cells to extravasate into other organs, blood vessels, lymph, or bone marrow and thereby reduce, decrease, or inhibit, or slow the progression of the cancer, including reducing, decreasing, inhibiting, or slowing metastasis.

As described herein, at least one (i.e., one or more) of the above described agents (e.g., compounds of formula (I)) may be administered in combination with at least one (i.e., one or more) additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with an E-selectin antagonist therapeutic agent described herein. Examples of inhibitors of PI3K include the compound named by Exelixis as “XL499.” Examples of VEGF inhibitors include the compound called “cabo” (previously known as XL184). Many other chemotherapeutics are small organic molecules. As understood by a person skilled in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

An E-selectin antagonist, such as a glycomimetic compound described herein may function independent of the anti-cancer agent, or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner)†) or maintaining survival of hematopoietic stem cells (HSC) in a subject who is treated with or will be treated with a chemotherapeutic drug(s) or radioactive therapy, respectively, comprising administering one or more of the E-selectin antagonist glycomimetic compounds described herein. In certain embodiments, the subject receives or will receive both chemotherapy and radiation therapy. Also, provided herein is a method for reducing (i.e., reducing, inhibiting, diminishing in a statistically or biologically significant manner) chemosensitivity or radiosensitivity of hematopoietic stem cells (HSC) to the chemotherapeutic drug(s) or radioactive therapy, respectively, in a subject. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy or radiotherapy or a combination of both chemotherapy and radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugiyama et al., Immunity 25:977-88 (2006); Mendez-Ferrer et al., Nature 466:829-34 (2010); Butler et al., Cell Stem Cell 6:251-64 (2010)). A recent study describes that E-selectin promotes HSC proliferation and is an important component of the vascular niche (see, e.g., Winkler et al., Nature Medicine published online 21 Oct. 2012; doi:10.1038/nm.2969). Deletion or inhibition of E-selectin enhanced HSC survival in mice that were treated with chemotherapeutic agents or radiotherapy and accelerated blood neutrophil recovery (see, e.g., Winkler et al., supra).

In addition, the administration of one or more of the E-selectin antagonist agents described herein may be in conjunction with one or more other therapies, e.g., for reducing toxicities of therapy. For example, at least one (i.e., one or more) palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one E-selectin antagonist described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g., AML, ALL, CLL, and CML), lymphoma (e.g., Hodgkin lymphoma and non-Hodgkin lymphoma), and myeloma (e.g., multiple myeloma). As used herein, the term cancer cells includes mature, progenitor and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure block infiltration of disseminated cancer cells into bone marrow. A variety of individuals may benefit from treatment with the compounds. Examples of such individuals include individuals with a cancer type having a propensity to migrate to bone where the tumor is still localized or the tumor is disseminated but not yet infiltrated bone, or where individuals with such a cancer type are in remission.

The cancer patient population most likely to respond to treatment using the E-selectin antagonist agents (e.g., compounds of formula (I)) described herein can be identified based on the mechanism of action of E-selectin. That is, patients may be selected that express a highly active E-selectin as determined by the genetic polymorphism for E-selectin of S128R (Alessandro et al., Int. J. Cancer 121:528-535, 2007). In addition, patients for treatment by the agents described herein may also selected based on elevated expression of the E-selectin binding ligands (sialyl Lea and sialyl Lex) as determined by antibodies directed against cancer-associated antigens CA-19-9 (Zheng et al., World J. Gastroenterol. 7:431-434, 2001) and CD65. In addition, antibodies HECA-452 and FH-6 which recognize similar carbohydrate ligands of E-selectin may also be used in a diagnostic assay to select the cancer patient population most likely to respond to this treatment.

### Methods for Treating or Preventing Thrombus Formation

The E-selectin antagonist agents described herein (compounds of structure (I)) may be useful in methods for preventing (i.e., reducing the likelihood of occurrence or recurrence of) and/or treating thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers and adults. An individual may have a hereditary predisposition to thrombosis. Thrombosis may be initiated, for example, due to a medical condition (such as cancer or pregnancy), a medical procedure (such as surgery) or an environmental condition (such as prolonged immobility). Other individuals at risk for thrombus formation include those who have previously presented with a thrombus.

An E-selectin antagonist therapeutic agent described herein is used to treat individuals undergoing thrombosis or who are at risk of a thrombotic event occurring. Such individuals may or may not have a risk of bleeding. In an embodiment, the individual has a risk of bleeding. In an embodiment, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LMW heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contract, administration of the E-selectin antagonist compounds of formula I significantly reduces the time to clotting than occurs when LMW heparin is administered, and thus provide a significant improvement in reducing bleeding time compared with LMW heparin. Accordingly, the E-selectin antagonists agents described herein are not only useful for treating a patient for whom the risk of bleeding is not significant, but also are useful in when the risk of bleeding is significant and the use of anti-thrombosis agents with anti-coagulant properties (such as LMW heparin) is contraindicated.

At least one (i.e., one or more) of the above described agents (i.e., an E-selectin antagonist, such as a glycomimetic compound of formula (I)) may be administered in combination with at least one (i.e., one or more) additional anti-thrombosis agent. An E-selectin antagonist agent described herein may function independent of the anti-thrombosis agent, or may function in coordination with the anti-thrombosis agent. In addition, the administration of one or more of the E-selectin antagonist agents described herein may be in conjunction with one or more other therapies, e.g., for reducing toxicities of therapy. For example, at least one palliative agent to counteract (at least in part) a side effect of therapy may be administered. Agents (chemical or biological) that promote recovery, or counteract side effects of administration of antibiotics or corticosteroids, are examples of such palliative agents. At least one agent described herein may be administered before, after or concurrently with administration of at least one additional anti-thrombosis agent or at least one palliative agent to reduce a side effect of therapy. Where administration is concurrent, the combination may be administered from a single container or two (or more) separate containers.

### Pharmaceutical Compositions and Methods of Using Pharmaceutical Compositions

Also provided herein are pharmaceutical compositions that comprise any one or more of the E-selectin antagonist agents described herein, such as one or more of the glycomimetic compounds of formula I (and substructures and specific structures thereof) described herein. For example, in one embodiment a composition comprising any of the above compounds of structure (I) and a pharmaceutically acceptable carrier, diluent or excipient is provided.

In other embodiments, a composition comprising a compound of formula (Ik), a compound of formula (IId) and a pharmaceutically acceptable carrier, diluent or excipient is provided. In certain embodiments of such compositions the compound of formula (Ik) has the following structure:

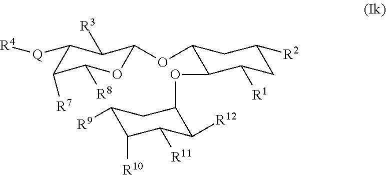

or a pharmaceutically acceptable salt, stereoisomer, tautomer, prodrug or solvate thereof, wherein:

Q is —O—, —S— or —CH₂—;

R¹ is C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₃-C₆ cycloalkyl, or C₃-C₆ halocycloalkyl;

R² is H, -L¹-C₁-C₂₅ alkyl, -L¹-C₂-C₂₅ alkenyl, -L¹-C₂-C₂₅ alkynyl, -L¹-C₁-C₂₅ haloalkyl, -L¹-C₂-C₂₅ haloalkenyl or -L¹-C₂-C₂₅ haloalkynyl;

R³ is —OC(═O)aryl or —NHC(═O)R³;

R⁴ is aryl, aralkyl or has the following structure:

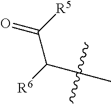

R⁵ is —OR¹⁴, —NHOR¹⁵, or —N(R¹⁵)(R¹⁶);

R⁶ is C₁-C₈ haloalkyl, C₂-C₈ haloalkenyl, C₂-C₈ haloalkynyl, cycloalkylalkyl or halocycloalkylalkyl;

R⁷, R¹⁰, R¹¹ and R¹² are each independently —OH, halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R⁸ is —CH₂OH, C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R⁹ and R¹³ are each independently C₁-C₈ alkyl, C₂-C₈ alkenyl, C₂-C₈ alkynyl, C₁-C₈ haloalkyl, C₂-C₈ haloalkenyl or C₂-C₈ haloalkynyl;

R¹⁴ is H, C₁-C₂₅ alkyl, C₂-C₂₅ alkenyl, C₂-C₂₅ alkynyl, C₂-C₂₅ haloalkyl, C₂-C₂₅ haloalkenyl or C₂-C₂₅ haloalkynyl;

R¹⁵ and R¹⁶ are each independently H, C₂-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl; and

L¹ is an optional linker,

and the compound of formula (IId) has the following structure:

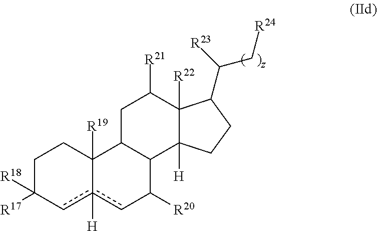

or a pharmaceutically acceptable salt, stereoisomer, tautomer, prodrug or solvate thereof, wherein:

R¹⁷ is H, —OH, —N₃, —OR²⁵, —N(R²⁷)(R²⁸), —NHC(═O)R²⁷, —C(═O)N(R²⁷)(R²⁸), —OC(═O)Ar, —NC(═O)Ar, —OC(═O)OR²⁹ or —OC(═O)R²⁹ or R¹⁷ joins with R^{1′} to form oxo or ═NCH₂Ar;

R¹⁸ is H or —NH₂ or R¹⁸ joins with R¹⁷ to form oxo or ═NCH₂Ar;

R¹⁹, R²² and R²³ are each independently H, C₁-C₈ alkyl;

R²⁰ and R²¹ are each independently H, —OH or —OR²⁵;

R²⁴ is C(═O)OR³⁰, —CH₂OR²⁹, —C(═O)N(R³¹)(R³²), —C(═O)SR³⁰, —CH₂S(O)ₚ—SR³⁰, —CH₂N(R²⁷)(R²⁸) or —CH₂S(O)ₚ—SR³⁰;

R²⁵ is a monosaccharide or an oligosaccharide comprising from 2-10 monosaccharides, wherein each glycosidic linkage at any anomeric carbon in the monosaccharide or oligosaccharide independently has the alpha or beta configuration;

R²⁷ and R²⁸ are each independently H, C₁-C₄ alkyl, C₃-C₇ cycloalkyl, C₄-C₁₀ alkylcycloalkyl or aryl or R²⁷ joins with R²⁸ to form a 4, 5, 6 or 7-membered heterocycle;

R²⁹ is H or C₁-C₃ alkyl;

R³⁰ is H C₁-C₁₀ alkyl, C₂-C₁₀ alkenyl, C₂-C₁₀ alkynyl, aryl or aralkyl;

R³¹ and R³² are each independently H, C₁-C₄ alkyl, C₃-C₇ cycloalkyl, C₄-C₁₀ alkylcycloalkyl, polyalkylamine or aryl or R³¹ joins with R³² to form a mono, bi or tricyclic heterocycle containing from 1 to 5 nitrogen atom;

Ar is optionally substituted phenyl;
p and z are each independently 0, 1 or 2; and

a dashed line indicate an optional double bond,

wherein all valences are satisfied.

In some embodiments, the compound of structure (Ik) has the following structure (II):

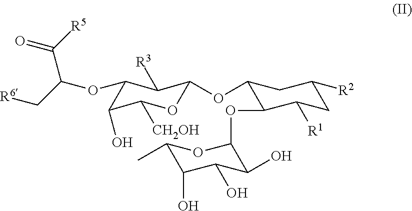

wherein R^{6′} is C₁-C₇ haloalkyl, C₂-C₇ haloalkenyl, C₂-C₇ haloalkynyl, C₃-C₆ cycloalkyl or C₃-C₆ halocycloalkyl.

In some other embodiments, R¹⁷ is —OH, R¹⁸ is H and R²⁴ is —CO₂H. In still other embodiments, R¹⁷ is —OH, R¹⁸ is H and R²⁴ is —CO₂CH₃.

In some other exemplary embodiments, R¹⁸ is H, R¹⁹ is H and R²⁴ is —CO₂H.

In still other embodiments, R¹⁷ is H, R¹⁸ is —NH₂ and R²⁴ is —CO₂CH₃.

In some other embodiments, at least one of R¹⁷, R²⁰ or R²¹ is —OR²⁵. For example, in some embodiments each of R²⁰ and R²¹ is —OR²⁵.

In some certain other embodiments, R² is alpha or beta glucose.

The compounds described herein may be formulated in a pharmaceutical composition for use in treatment or preventive (or prophylactic) treatment (e.g., reducing the likelihood of occurrence or of exacerbation of a disease, or of one or more symptoms of the disease). The methods and excipients described herein are exemplary and are in no way limiting.

In pharmaceutical dosage forms, any one or more of the glycomimetic compounds of formula I, substructures and specific structures described herein may be administered in the form of a pharmaceutically acceptable derivative, such as a salt, or they may also be used alone or in appropriate association, as well as in combination, with other pharmaceutically active compounds.

An effective amount or therapeutically effective amount refers to an amount of a glycomimetic compound or a composition comprising one or more compounds, or other E-selectin antagonist agent, that when administered to a subject, either as a single dose or as part of a series of doses, is effective to produce a desired therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, or blood volume of the subject. In general, the amount of a glycomimetic compound described herein, that is present in a dose, ranges from about 0.01 μg to about 1000 μg per kg weight of the host. In general, the amount of a compound of structure (I), as described herein, present in a dose, also ranges from about 0.01 μg to about 1000 μg per kg of subject. The use of the minimum dose that is sufficient to provide effective therapy is usually preferred. Subjects may generally be monitored for therapeutic effectiveness using assays suitable for the disease or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease or disorder may be determined according to parameters understood by a person of ordinary skill in the medical art.

Pharmaceutical compositions may be administered in a manner appropriate to the disease or disorder to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the pharmaceutical composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively deliver an effective amount of the compound. Such administrative routes include, for example, topical, oral, nasal, intrathecal, enteral, buccal, sublingual, transdermal, rectal, vaginal, intraocular, subconjunctival, sublingual or parenteral administration, including subcutaneous, intravenous, intramuscular, intrasternal, intracavernous, intrameatal or intraurethral injection or infusion. Compositions administered by these routes of administration and others are described in greater detail herein.

A pharmaceutical composition may be a sterile aqueous or sterile non-aqueous solution, suspension or emulsion, which additionally comprises a physiologically acceptable excipient (pharmaceutically acceptable or suitable excipient or carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds described herein may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient or carrier known to those of ordinary skill in the art for use in pharmaceutical compositions may be employed in the compositions described herein. Excipients for therapeutic use are well known, and are described, for example, in Remington: The Science and Practice of Pharmacy (Gennaro, 21^{st} Ed. Mack Pub. Co., Easton, Pa. (2005)). In general, the type of excipient is selected based on the mode of administration, as well as the chemical composition of the active ingredient(s). Pharmaceutical compositions may be formulated for the particular mode of administration. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

For oral formulations, at least one of the E-selectin antagonist agents described herein can be used alone or in combination with appropriate additives to make tablets, powders, granules or capsules, for example, with any one or more conventional additives, disintegrators, lubricants, and if desired, diluents, buffering agents, moistening agents, preservatives, coloring agents, and flavoring agents. The compositions may be formulated to include a buffering agent to provide for protection of the active ingredient from low pH of the gastric environment and/or an enteric coating. A composition may be formulated for oral delivery with a flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The compositions may be formulated into pressurized acceptable propellants such as dichlorodifluoromethane, propane, nitrogen and the like.

Any one or more of the therapeutic molecules described herein may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneras et al., J. Pharm. Pharmacol. 54:499-508 (2002); Karande et al., Pharm. Res. 19:655-60 (2002); Vaddi et al., Int. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits with unit doses of one or more of the compounds, described herein, usually in oral or injectable doses, are provided. Such kits may include a container containing the unit dose, an informational package insert describing the use and attendant benefits of the therapeutic in treating the pathological condition of interest, and optionally an appliance or device for delivery of the composition.

### EXAMPLES

### Example 1

### Synthesis of E-Selectin Inhibitor

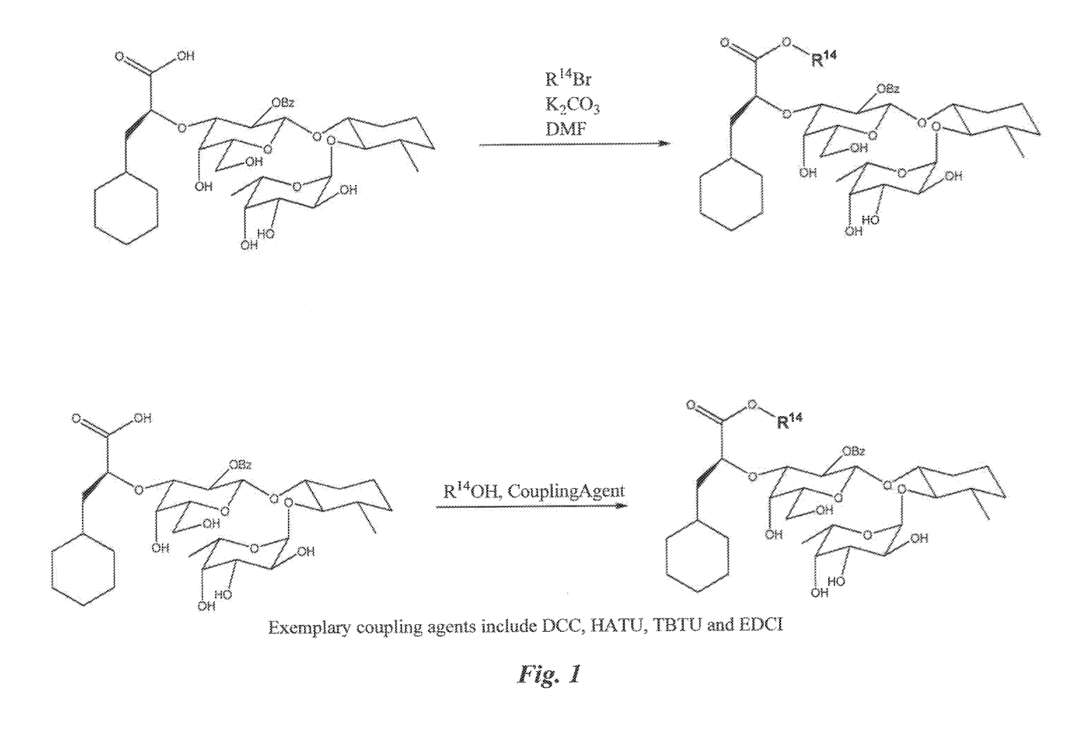

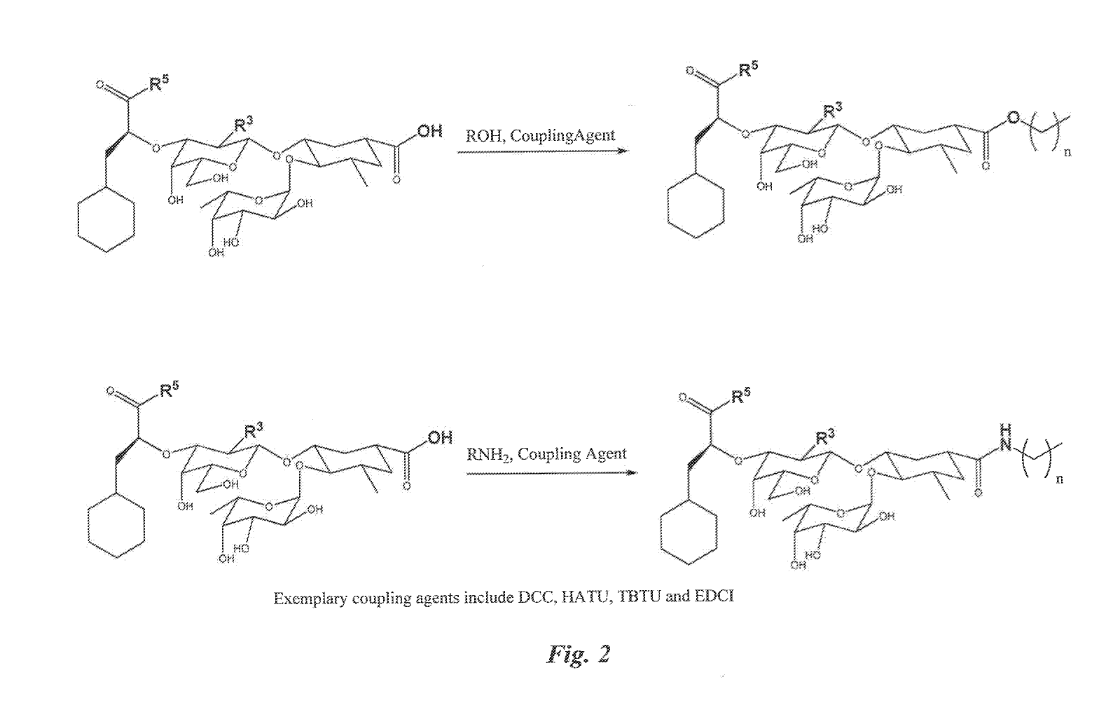

Exemplary glycomimetic compounds of formula (I) were synthesized as described in FIGS. 1 and 2 and in this Example as shown in the following exemplary synthesis schemes.

### Summary of Synthetic Scheme for Compound 21 (Common Intermediate for Compound 23 and Compound 25)

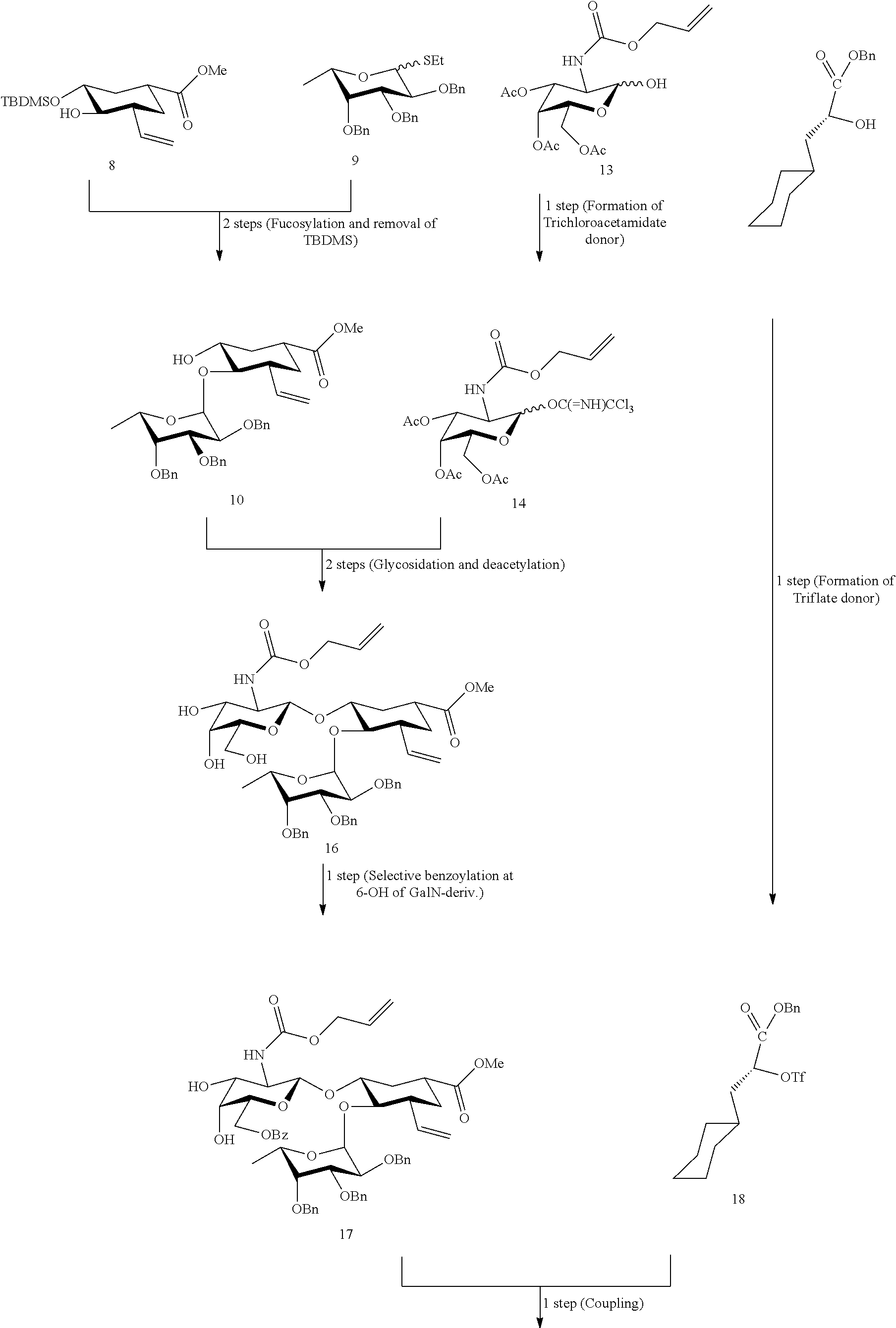

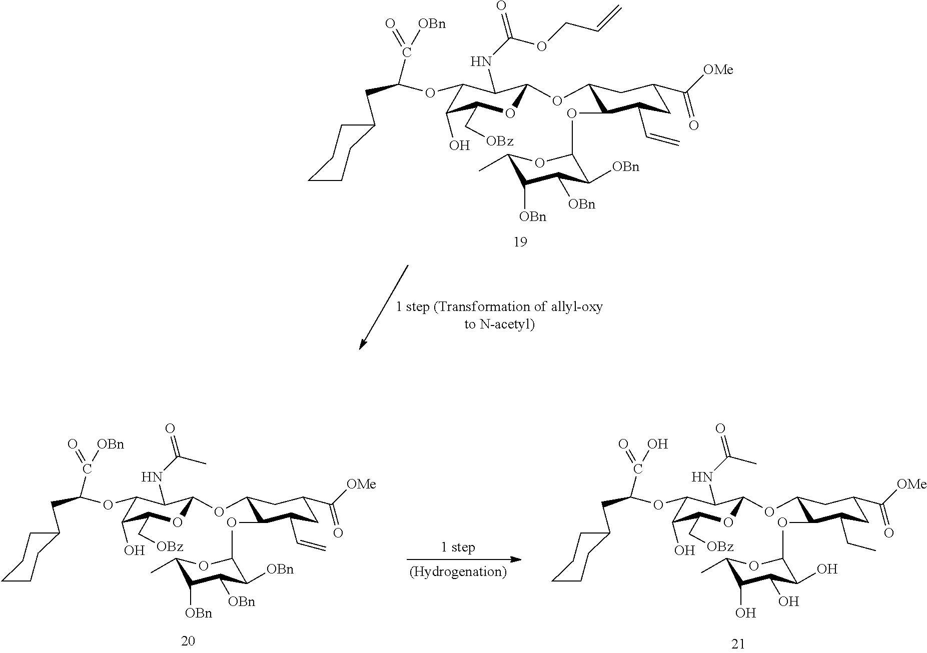

### Synthesis of Compound 8

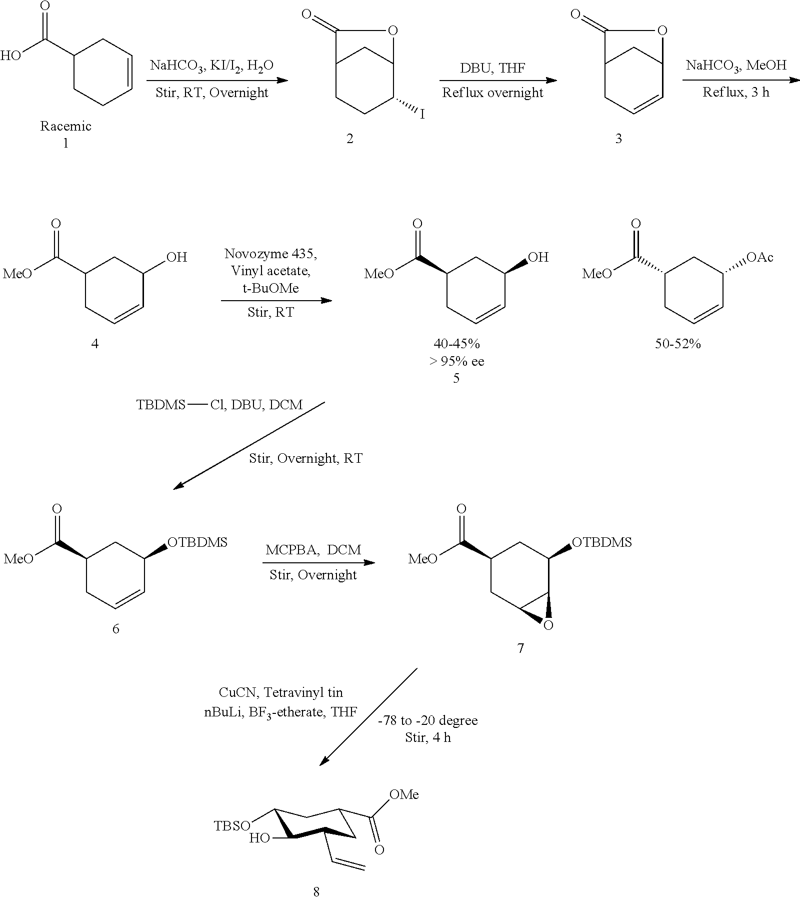

### Synthesis of Compound 10

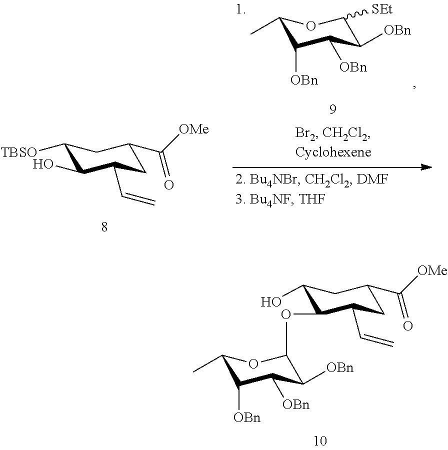

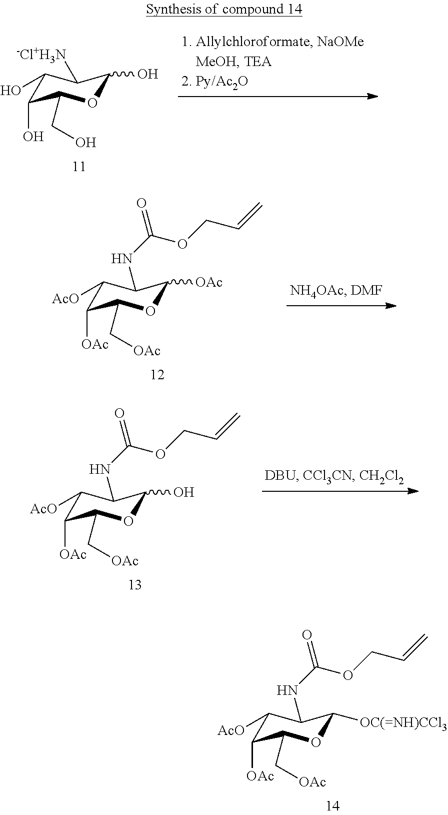

### Synthesis of Compound 20

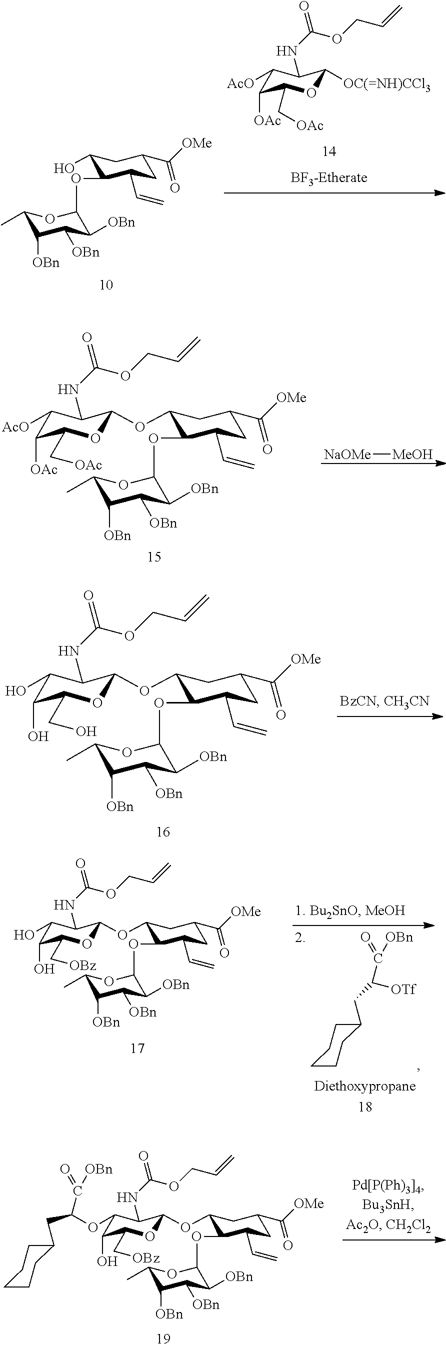

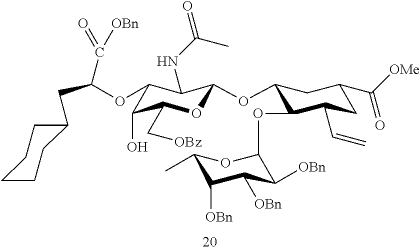

### Synthesis of Compound 23

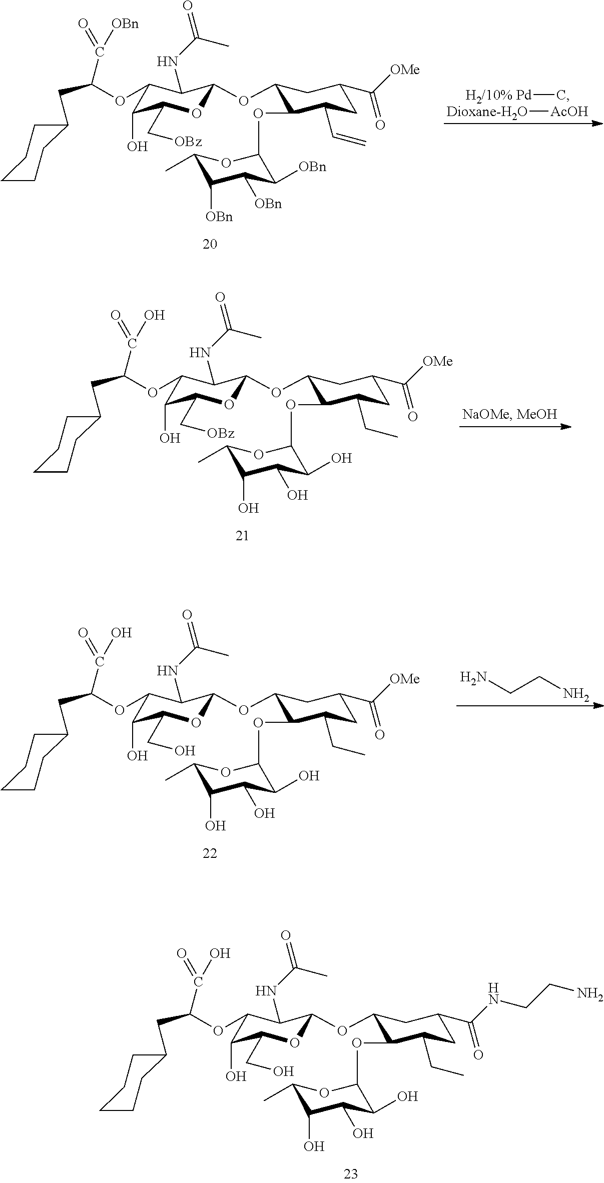

### Synthesis of Compound 25

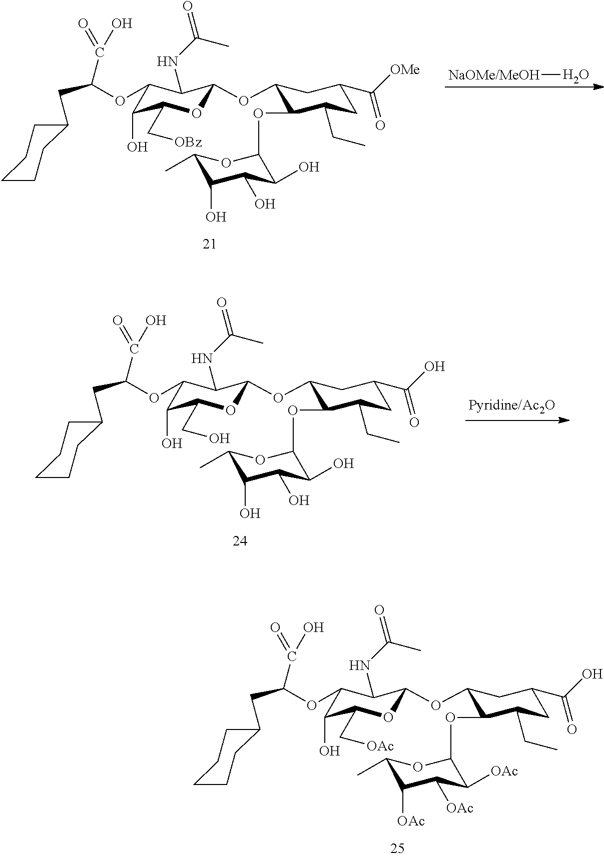

### Synthesis of Compound 31

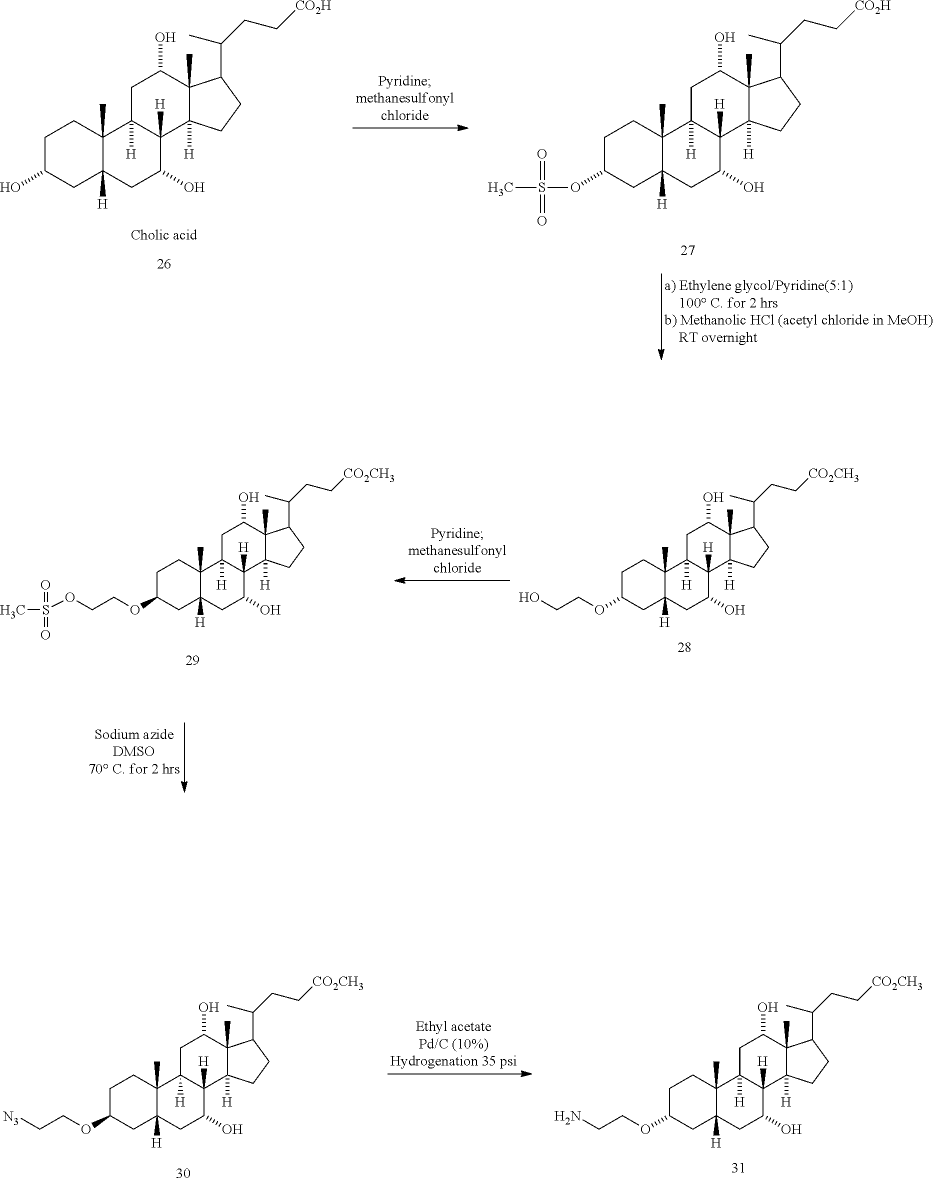

### Synthesis of Compound 33:

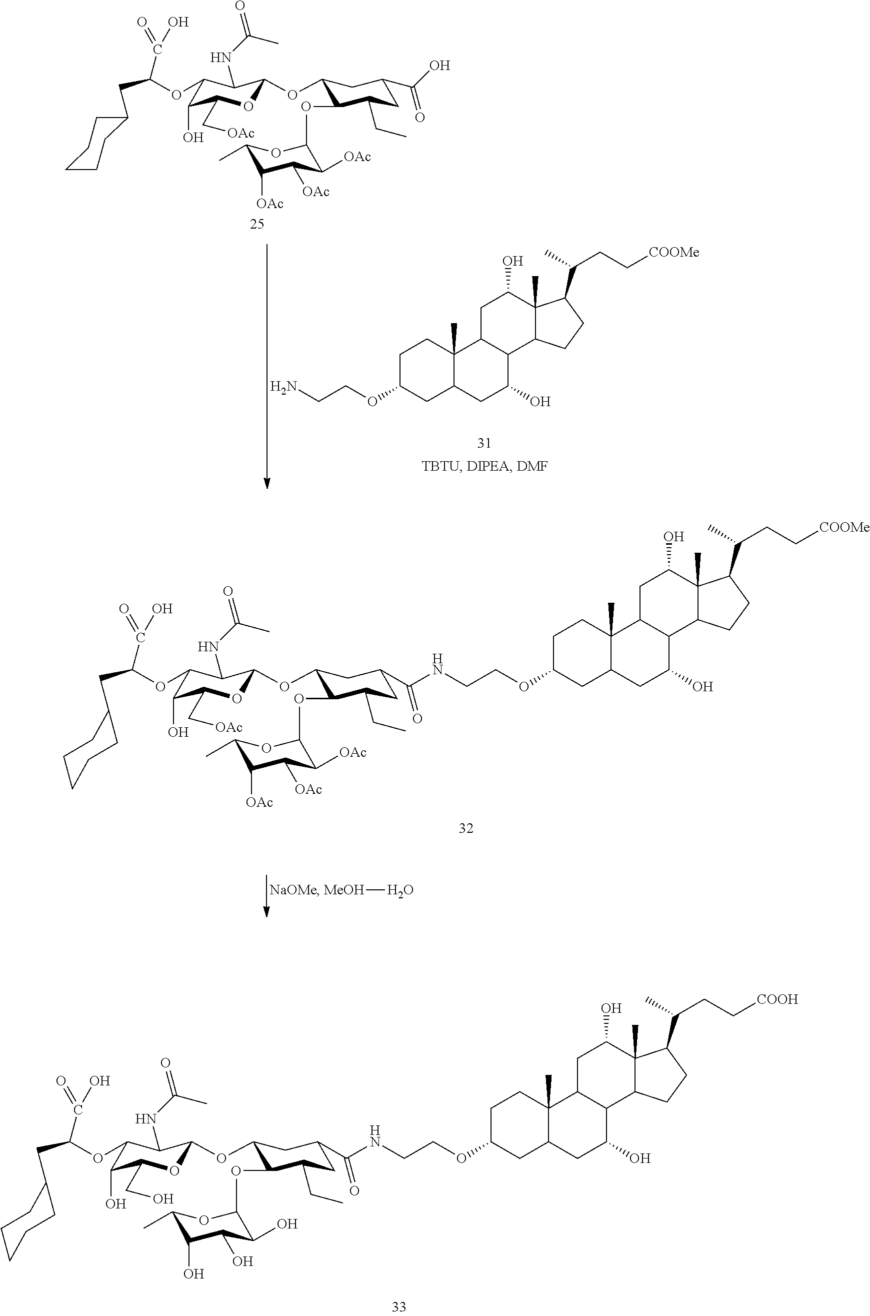

### Synthesis of Compound 35:

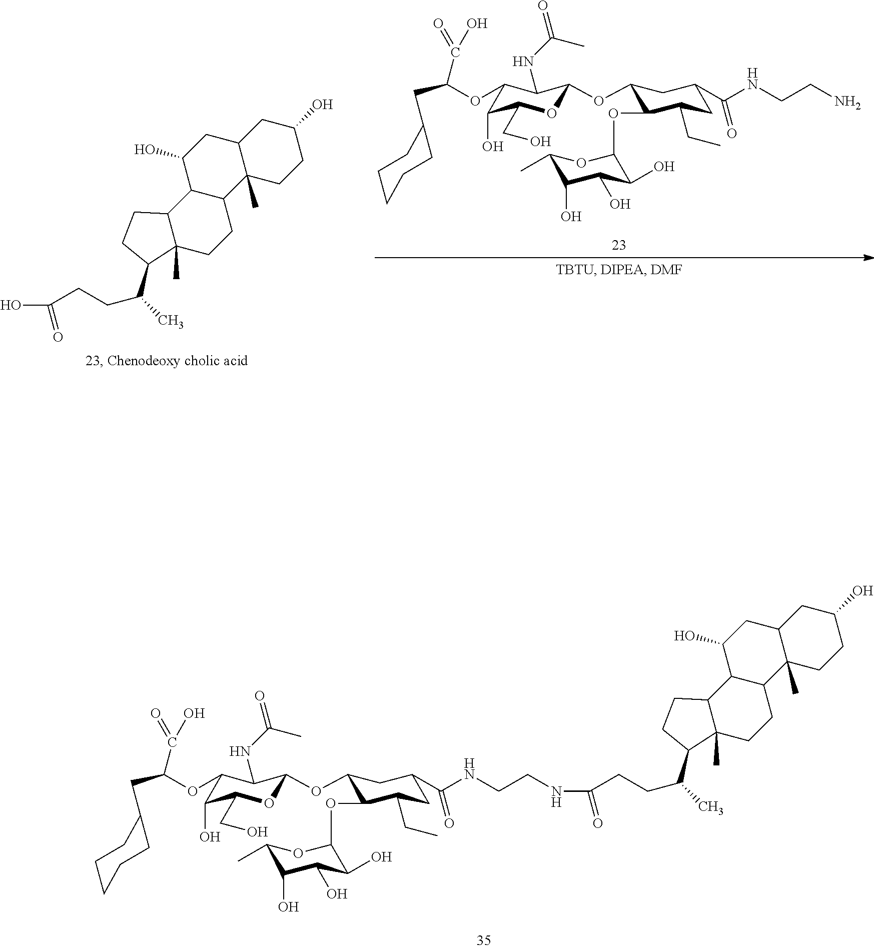

### Synthesis of Compound 2:

Compound 1 (60 g) was suspended in H₂O (800 ml) and cooled to 0° C. Solid NaHCO₃ (120 g) was added in portion with stirring and then a solution of KI (474.3 g) and I₂ (127 g) in H₂O (800 ml) was added with stirring. Reaction mixture was stirred at room temperature overnight in the dark. Reaction mixture was extracted with CH₂Cl₂ (3×500 ml). Organic layer was washed with Na₂S₂O₃ solution (2×500 ml) and then combined aqueous layer was extracted with CH₂Cl₂ (2×300 ml). Organic layers (2100 ml) were combined and washed with cold H₂O (1×500 ml) and cold brine (1×500 ml). Organic layer was dried over Na₂SO₄, filtered and concentrated to dryness to give compound 2 as light yellow crystals (119 g). Purity: >95% by TLC.

### Synthesis of Compound 3:

To a solution of compound 2 (119 g) in THF (1600 ml) was added DBU (119 ml) with stirring at room temperature, and the reaction mixture was gently refluxed overnight with stirring. Some precipitate formed and TLC showed no starting material left. Reaction mixture was concentrated to dryness and dissolved in EtOAc (300 ml), washed with 0.5M HCl (200 ml) until pH 2-3 of the aqueous layer and H₂O (200 ml). Aqueous layers were combined and were extracted with EtOAc (3×200 ml). Combined organic layers (900 ml) were washed with brine, dried (Na₂SO₄), filtered and concentrated to dryness to give compound 3 (58 g). Purity: >95% by TLC

### Synthesis of Compound 4:

To a solution of compound 3 (58 g) in MeOH (800 ml) was added NaHCO₃ (47 g) with stirring. The reaction mixture was stirred under gentle reflux for 3 h, and then cooled to room temperature, filtered and concentrated to dryness. The residue was dissolved in EtOAc (300 ml) and washed with H₂O. Aqueous layer was extracted with EtOAc (3×100 ml). Combined organic layers (600 ml) were washed with 0.5M HCl (200 ml), H₂O (100 ml), and brine (100 ml), dried (Na₂SO₄), then filtered, and concentrated to dryness. The residue was purified by column chromatography (SiO₂, Hexanes-EtOAc 3:1→3:2) to give compound 4 (54 g). Purity: >95% by TLC

### Synthesis of Compound 5:

Compound 4 (31 g) was dissolved in tBuOMe (620 ml) and vinylacetate (166 ml) was added with vigorous stirring. Novozyme 435 (1.4 g) was added and vigorous stirring was continued for 5.5 h. Reaction mixture was filtered and stored at −20° C. Next morning another batch of Novozyme 435 resin (1.4 g) was added and stirred vigorously for 8 h. Resin was filtered and concentrated to dryness. Oily residue was purified by CombiFlash® system (silica) using 0→50% EtOAc/Hexanes to give compound 5 (13.0 g).

### Synthesis of Compound 6:

Compound 5 (13.5 g) was dissolved in CH₂Cl₂ (300 ml) under argon and TBDMS-Cl (26.4 g) was added with stirring at room temperature under argon. DBU (32.4) was added and stirring was continued for overnight at room temperature under argon. MeOH (30 ml) was added and washed with cold saturated solution of NaHCO₃ (200 mil), brine (150 ml). The organic layer was dried (Na2SO4), filtered and concentrated to dryness. The residue was purified by CombiFlash® system (SiO₂) using solvent EtOAc-Hexanes (0-15%) to give compound 6 (18 g). Purity >95% by TLC.

### Synthesis of Compound 7:

Compound 6 (12 g) was dissolved in CH₂Cl₂ (400 ml) and cooled to 0° C. m-chloroperbenzoic acid (77%, 19 g) was added and the solution was stirred for few hours during which the temperature of the reaction mixture reached to room temperature. The stirring was continued overnight at room temperature. CH₂Cl₂ (300 ml) was added and washed with cold saturated solution of NaHCO₃ (3×400 ml), brine (cold), dried (Na2SO4), filtered, and concentrated to dryness. The residue was purified by CombiFlash® system (SiO2) using EtOAc-Hexanes (0→30%) to give compound 7 (9 g). Purity: >95% by TLC.

### Synthesis of Compound 8:

All operation of this step were done in argon atmosphere. CuCN (9.42 g) was dried at 160° C. under vacuum for 40 min, cooled down to room temperature and suspended in THF (80 ml). The mixture was cooled down to −78° C. During this time, tetravinyltin (12 ml) and n-BuLi in hexane (2.5M, 100 ml) were reacted for 30 min at 0° C. in THF (30 ml). This solution was added to the mixture of CuCN in THF, and the resulting mixture was stirred for 30 min. at −20° C. The mixture was then cooled to −78° C. to which was added a solution of freshly distilled BF₃.Et2O (6 ml) in THF (20 ml). The mixture is stirred for 20 min. at −78° C. Compound 7 (5 g) in THF (40 ml) was added and the reaction mixture was stirred at −78° C. for 5 h. MeOH (7 ml) and Et₃N (3 ml) were added and the mixture was concentrated to dryness. The residue was dissolved in EtOAc (200 ml) and washed with saturated solution of NaHCO₃ (2×100 ml), brine (100 ml), dried (Na₂SO₄), filtered, and concentrated to dryness. The residue was purified by CombiFlash® system (SiO2) using solvent EtOAc-Hexanes (0→5%) to give compound 8 (2.5 g).

### Synthesis of Compound 10:

Compound 8 (2.25 g, 7 mmol) was dissolved in toluene (7 ml) and solvent is evaporated off. The process repeated twice and finally dried under vacuum for 15 min. The residue was dissolved in anhydrous CH₂Cl₂ (45 ml) and DMF (45 ml) was added. The solution was stirred under argon at room temperature and molecular sieves (3 g, 4 Å, powdered and flamed dried) was added. Et₄NBr (3.3 g, 15.7 mmol, 2.2 equivalents, dried at 200 OC for 2 h) was added and the stirring was continued for h at room temperature under argon.

Compound 9 (5.13 g, 10 mmol, 1.42 equivalents) was co-evaporated with toluene (3×20 ml) and dried under vacuum and dissolved in CH₂Cl₂ (45 ml). The reaction mixture was placed in an ice-bath and stirred for 10 min. To this solution was added Br₂ (0.8 ml, 15 mmol, 1.5 equivalents) drop-wise with stirring in the ice-bath. Stirring was continued for 40 min at the same temperature. Ice-bath was removed and cyclohexene (2.1 ml) was added slowly with stirring after 10 min. The reaction mixture was stirred for 10 min. and added slowly to the reaction mixture above with stirring at room temperature under argon. Stirring was continued for 17 h and then pyridine (4 ml) was added, filtered and the filtrate was concentrated to dryness. The residue was dissolve in CH₂Cl₂ (100 ml) and transferred to a separatory funnel. The organic layer was washed with cold brine (2×75 ml), dried (Na₂SO₄), filtered and concentrated to dryness, co-evaporated with toluene (3×50 ml), and dried under vacuum. The residue was dissolved in THF (8 ml) and a solution of TBAF (1M in THF, 10 ml, 10 mmol, 1.42 equivalents) was added with stirring at room temperature. Stirring was continued for 15 h and solvent was evaporated off. The residue was dissolved in CH₂Cl₂ (100 ml) and transferred to a separatory funnel, washed with cold brine (2×75 ml), dried (Na₂SO₄), filtered, and concentrated to dryness. The residue was purified by column chromatography (Hexanes-Ethyl acetate from 100% hexanes to 70% hexanes in EtOAc) to give compound 10 (1.6 g, 2.59 mmol, 37% overall in two steps). TLC: 5% EtOAc in hexanes and 33% EtOAc in hexanes.

### Synthesis of Compound 12:

Commercially available compound 11 (10 g) was dried overnight under vacuum overnight and added to a solution of NaOMe (5M, 10 ml) in MeOH (200 ml) with stirring at room temperature under argon. Stirring was continued for overnight at room temperature argon to which was added Et₃N (7 ml) followed by allylchloroformate (3.5 ml) dropwise. Stirring was continued for 6 h at room temperature under argon. Reaction mixture was concentrated to dryness and dissolved in pyridine (100 ml). Ac₂O (50 ml) was added at room temperature under argon and stirred at room temperature for overnight. The reaction mixture was concentrated to dryness and purified by column chromatography on CombiFlash® system using EtOAc-Hexanes (0-100%). The desired fractions were collected and concentrated to dryness to give compound 12 (10.2 g).

### Synthesis of Compound 13:

Compound 12 (7.5 g) was dissolved in DMF (140 ml) and was added NH₄OAC (4.05 g) with stirring. Stirring was continued for overnight at room temperature under argon. Next day the reaction mixture was stirred at 50° C. under argon for 8 h. The reaction mixture was concentrated to dryness and the residue was dissolved in EtOAc (150 ml), washed with brine (100 ml), dried (Na₂SO4), filtered, and concentrated to dryness. The residue was purified by column chromatography (SiO₂, Hexanes-EtOAc 2:1→1:2) to give compound 13 (6 g).

### Synthesis of Compound 14:

Compound 13 (6 g) was dissolved in CH₂Cl₂ (50 ml) and as added CCl₃CN (6 ml) and DBU (0.5 ml). The reaction mixture was stirred at room temperature for 0.5 h, solvent was evaporated off and the residue was purified by column chromatography (silica gel) to give compound 14 (4.5 g).

### Synthesis of Compound 15:

Compound 10 (2 g) and compound 14 (2.1 g) were dissolved in CH₂Cl₂ (40 ml). To this solution were added molecular sieves (4 Å, 0.8 g) and stirred at room temperature for 30 min. The solution was then cooled to 0 OC and BF₃.Et₂O (0.25 ml dissolved in 5 ml) was added with stirring at 0 OC. The reaction mixture was stirred at 0° C. for 2 h. Et₃N (0.5 ml) was added, and the solvent is evaporated off. The residue was purified by column chromatography (silica gel) to give compound 15 (1.8 g).

### Synthesis of Compound 16:

Compound 15 (1.7 g) was treated with 0.01N NaOMe in MeOH (10 ml) for 2 h and neutralized with IR-120 (H⁺) resin, filtered, and concentrated to dryness to give compound 16 (1.25 g).

### Synthesis of Compound 17:

To a solution of compound 16 (1.2 g) in CH₃CN (30 ml) was added Et3N (0.28 ml) and cooled to 0° C. To this solution was added BzCN (0.35 mg in 10 ml CH₃CN) dropwise during 20 min at 0° C. The reaction mixture was stirred for 1 h at 0° C. and concentrated to dryness. The residue was purified by column chromatography (silica gel) to give compound 17 (0.95 g).

### Synthesis of Compound 19:

Compound 17 (0.9 g) was dissolved in MeOH (12 ml). To this solution was added Bu₂SnO (0.4 g) and the mixture as refluxed for 2 h. Solvent was evaporated off and the residual solvent was co-evaporated off with toluene 3 times. The residue was dissolved in dimethoxy ethane (15 ml). To this solution was added CsF (0.8 g) and compound 18 (2.1 g, synthesized as described previously, J. Med. Chem. 1999, 42, 4909). The reaction mixture was stirred overnight at room temperature, and the solvent was evaporated off. The residue was purified by column chromatography to give compound 19 (0.8 g).

### Synthesis of Compound 20:

Compound 19 (0.7 g) was dissolved in CH₂Cl₂ (20 ml). To this solution was added Pd(Ph)₄ (0.14 g), Bu₃SnH (0.15 ml), and Ac₂O (0.3 ml), and the reaction mixture was stirred at room temperature for h. Solvent was evaporated off and the residue was purified by column chromatography (silica gel) to give compound 20 (0.5 g).

### Synthesis of Compound 21:

To a solution of compound 20 (0.45 g) in dioxane-H₂O—AcOH (10:2:1, 2.6 ml) was added 10% Pd—C (0.15 g), and the reaction mixture was shaken at room temperature under positive pressure (20 psi) of hydrogen for 5 h. The solid was filtered off and the filtrate concentrated to dryness. The residue was purified by column chromatography (silica gel) to give compound 21 (0.3 g).

### Synthesis of Compound 22:

Compound 21 (0.28 g) was treated with 0.025N NaOMe in MeOH (5 ml) for 4 h, neutralized with IR-120 (H+) resin, filtered, and the filtrate was concentrated to dryness to give compound 22 (0.21 g).

### Synthesis of Compound 23:

Compound 22 (0.18 g) was dissolved in ethylenediamine (2 ml) and stirred at 80 OC for 8 h. Solvent was evaporated off and the residue was purified Sep-pak C18 cartridges to give compound 23 (0.15 g).

### Synthesis of Compound 24:

Compound 21 (0.2 g) was dissolved in methanol (5 ml) and was added slowly NaOMe/MeOH (25%, 0.15 ml)) with stirring at RT. Water (0.5 ml) was added into the reaction mixture and was stirred at 50° C. for 3 hrs, neutralized with acetic acid (0.070 ml), concentrated to dryness and purified by silica gel column (CH₂Cl₂; CH₂Cl₂/MeOH 10:1; 10:2; 10:3; CH₂Cl2/MeOH/H2O 13:6:1) to give compound 24 (0.16 g).

### Synthesis of Compound 25:

Compound 24 (0.16 g) was dissolved in pyridine/acetic anhydride (8 ml, 1:1) and stirred overnight at 55° C. The reaction mixture was concentrated to dryness and purified by silica gel column (CH₂Cl₂; CH₂Cl₂/MeOH 10:1; 10:2; 10:3; 10:4; 10:5) to give compound 25 (180 mg).

### Synthesis of Compound 27:

Commercially available compound 26 (10 g, Cholic acid) was dissolved in pyridine (50 m) and stirred at 0° C. Methanesulfonyl chloride (2.31 ml) was added drop-wise at 0° C. The reaction mixture was stirred at 0° C. for 30 mins and at RT for 2 hrs. The reaction mixture was poured into 300 ml of water/40 ml of conc. H₂SO₄ and extracted using ethyl acetate (3×300 ml). The combined organic layers were dried using Na₂SO₄, filtered and concentrated to dryness. The crude product 27 (13.5 g) was used for the next step without further purification. TLC solvent system, 20% MeOH in CH₂Cl₂.

### Synthesis of Compound 28:

Part a)

Crude product 27 (13.5 g) was dissolved in 57 ml of ethylene glycol (57 ml) and was added pyridine (11.5 ml) with stirring at 100° C. for 2 hrs. The reaction mixture was poured into 170 ml of water (170 ml)/conc. H2SO4 (11.5 ml) and extracted using ethyl acetate (3×300 ml). The combined organic layers were dried using Na2SO₄, filtered, and concentrated to dryness. TLC solvent system, 20% MeOH in CH₂Cl₂.

Part b)

The crude product from part a was dissolved in 125 ml of methanolic HCl (125 ml, prepared by drop-wise adding 50 ml of acetyl chloride to 500 ml of methanol) and stirred overnight at RT. The reaction mixture was poured into water (225 ml) and extracted using ether (3×250 ml). Combined organic layers were washed using saturated NaHCO₃ solution in water. Organic layer was dried using Na2SO₄, filtered, and concentrated to dryness. The residue was purified by silica gel column (ethyl acetate; ethyl acetate/MeOH 10:0.5; ethyl acetate/MeOH 10:1) to give compound 28 (5.2 g)

### Synthesis of Compound 29:

Compound 28 (5.2 g) was dissolved in pyridine (21 ml) and stirred at 0° C. Methanesulfonyl chloride (0.9 ml) was added drop-wise at 0° C. with stirring. The reaction mixture was stirred at 0° C. for 15 mins and at RT for 1 hr. The reaction mixture was poured into water (70 ml) and extracted using ethyl acetate (3×150 ml). The combined organic layers were dried using Na2SO4, filtered, and concentrated to dryness. The residue was purified by silica gel column (ethyl acetate/hexanes 1:1; 2:1; 3:1) to give compound 29 (4.5 g).

### Synthesis of Compound 30:

Compound 29 (4.5 g) was dissolved in dry DMSO (18 ml) to which was added sodium azide (0.6 g). The reaction mixture was at 70° C. for 2 hrs. The reaction mixture was poured into water (200 ml) and extracted using ethyl acetate (3×200 ml). The combined organic layers were dried using Na₂SO₄, filtered, and concentrated to dryness. The residue was co-evaporated with toluene and dried under high vacuum to give compound 30 (5.0 g) and used in the next step without further purification.

### Synthesis of Compound 31:

Compound 30 (crude, 2.5 g) was dissolved in ethyl acetate (40 ml) and was added 10% Pd/C (1.6 g). The reaction was stirred under hydrogen (35 psi) for 16 h. The catalyst was filtered off and the filtrate was concentrated to dryness. The residue was purified by silica gel column (ethyl acetate; ethyl acetate/MeOH 10:0.5; 10:1;10:2; 10:3; 10:4; 10:5) to give compound 31 (0.8 g).

### Synthesis Compound 32:

Compound 25 (0.050 g) and compound 31 (0.054 g) were combined together, co-evaporated with toluene (3 times), and dried under vacuum. The mixture was dissolved in DMF (2.5 ml) and stirred at RT. DIPEA (0.05 ml) was added, and the reaction mixture was placed in an ice bath. TBTU (0.026 g) was added into the reaction mixture and stirred at 0° C. for 10 mins and then at RT for 3 hrs. The reaction mixture was concentrated to dryness and the residue was purified by silica gel column (CH₂Cl₂; CH₂Cl₂/MeOH 10:1; 10:2; 10:3) to give compound 32 (60 mg).

### Synthesis of Compound 33:

Compound 32 (0.060 g) was dissolved in 3 ml of methanol (3 ml) to which was added NaOMe/MeOH (25%, 0.060 ml)). Water (0.3 ml) was added into the reaction mixture and was stirred overnight at RT, neutralized with acetic acid (0.030 ml), and concentrated to dryness. The residue was purified by sep-pak C-18 column (H₂O; H₂O/MeOH 10:1; 10:2; 10:3; 10:4; 10:5) to give compound 33 (22 mg).

### Synthesis of Compound 35:

Commercially available compound 34 (0.070 g) was dissolved in DMF (1.5 ml) to which was added DIPEA (0.062 ml) with stirring. The reaction mixture was cooled to 0° C., and TBTU (0.051 g) was added. Stirring was continued for 10 min at 0° C. To this reaction mixture was added compound 23 (0.080 g) and stirred at room temperature for 2 h. Solvent was evaporated off under reduced pressure and the residue was purified by reverse phase HPLC to give compound 35 (0.060 g).

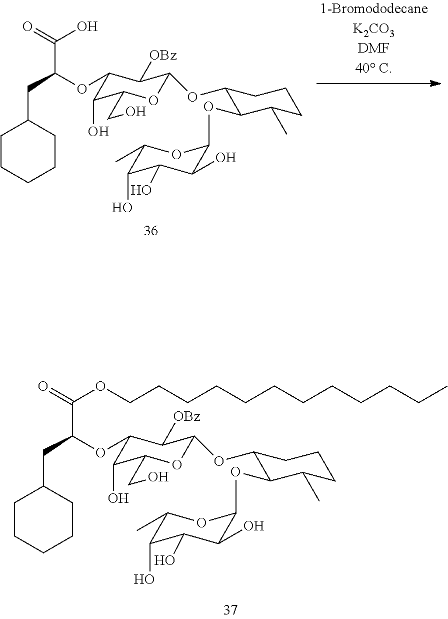

### Synthesis of Compound 37:

To a solution of carboxylic acid 36 (23 mg, 0.032 mmol) dissolved in DMF (0.3 mL) was added powdered potassium carbonate (50 mg) and 1-bromododecane (0.1 mL, 0.42 mmol). The reaction mixture was stirred overnight at 40° C. After cooling to room temperature, the reaction mixture was filtered through Celite then concentrated in vacuo. The reaction mixture was separated via CombiFlash® using a 4 g silica cartridge and eluting with 95/5 dichloromethane/methanol to afford 24 mg of compound 37 as a white solid. ¹H NMR (400 MHz, DMSO d-6) d 8.00 (2H, d, J=7.2 Hz), 7.66 (1H, t, J=7.2 Hz), 7.54 (2H, t, J=7.6 Hz), 5.27 (1H, dd, J=9.9 and 9.6 Hz), 4.65-4.58 (4H, m), 4.29 (1H, d, J=3.2 Hz), 4.21 (1H, d, 6.0 Hz), 4.18 (1H, d, J=6.8 Hz), 4.139-4.087 (2H, m), 4.06-4.00 (1H, dt, J=6.4 Hz), 3.93 (1H, br s), 3.64 (1H, dd, J=9.6 and 2.4), 3.58-3.47 (3H, m), 3.45-3.38 (4H, m), 2.95 (1H, t, J=8.4 Hz), 1.87 (1H, d, J=9.2 Hz), 1.55 (1H, m), 1.43-1.23 (24H, m), 1.11 (3H, d, J=6.81z), 0.96 (3H, d, J=6.4 Hz).

The following syntheses are performed to prove difluorolactates as an exemplary facial amphiphile.

### Difluorolactates

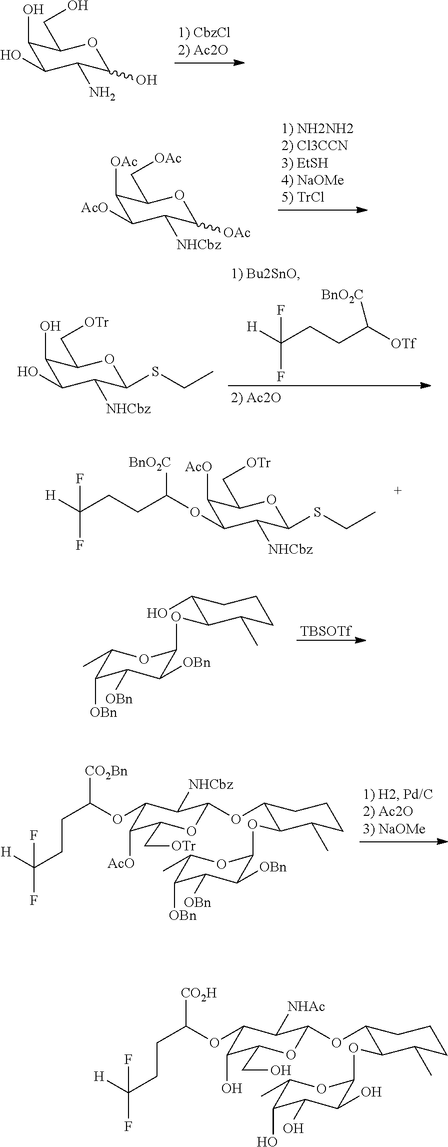

### Facial Amphiphile

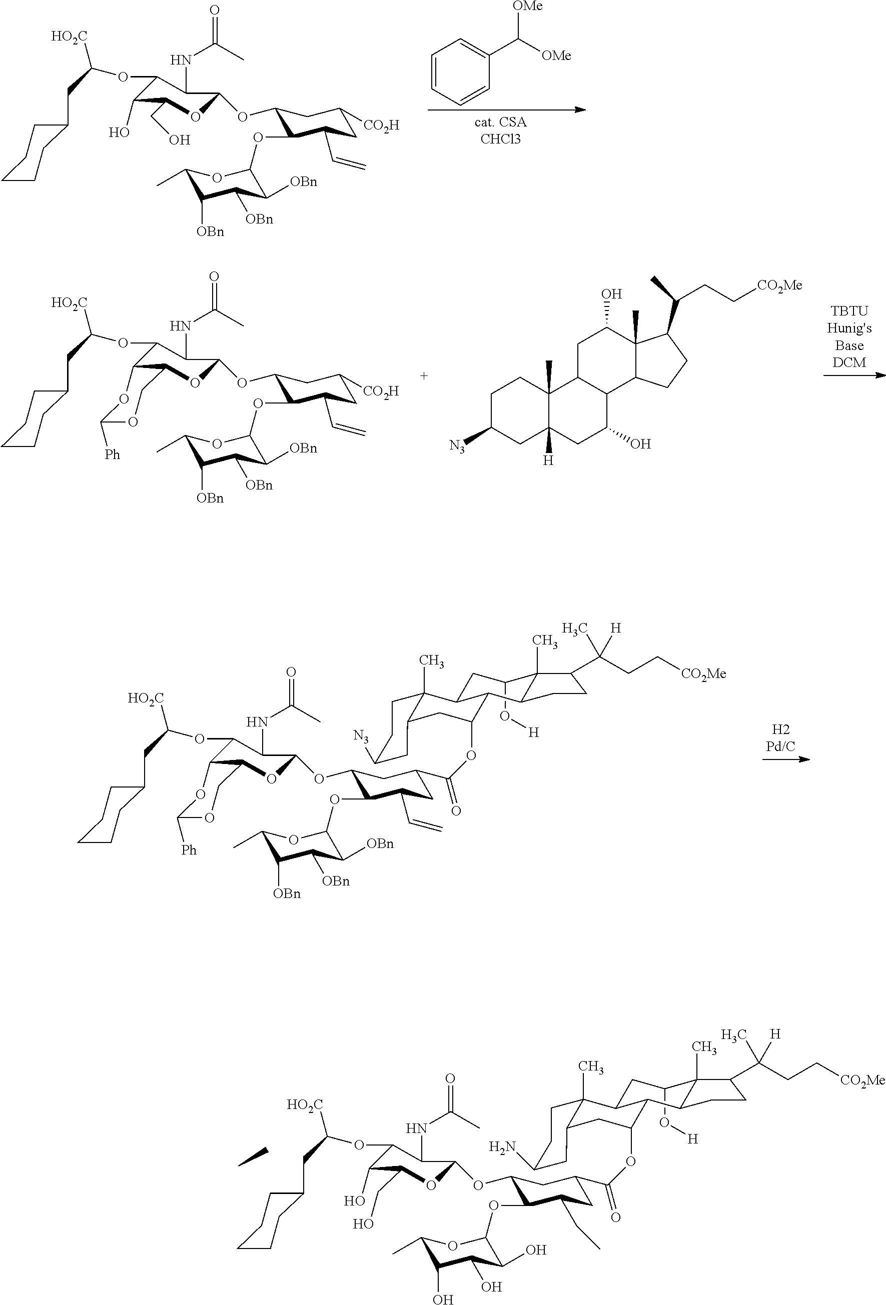

### Example 2

### E-Selectin Activity

### Binding Assay

The inhibition assay to screen and characterize glycomimetic antagonists of E-selectin is a competitive binding assay, from which IC₅₀ values may be determined. E-selectin/Ig chimera was immobilized in 96 well microtiter plates by incubation at 37° C. for 2 hours. To reduce nonspecific binding, bovine serum albumin was added to each well and incubated at room temperature for 2 hours. The plate was washed and serial dilutions of the test compounds were added to the wells in the presence of conjugates of biotinylated, sLe^{a} polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramethylbenzidine (TMB) was added. After 3 minutes, the enzyme reaction was stopped by the addition of H₃PO₄, and the absorbance of light at a wavelength of 450 nm was determined. The concentration of test compound required to inhibit binding by 50% was determined and reported as the IC₅₀ value for each glycomimetic E-selectin antagonist as shown in the table below. IC₅₀ values for exemplary compounds disclosed herein are provided in the following table.

E-Selectin Antagonist Activity of Glycomimetic Compounds

| Compound | rIC50 |
|---|---|
| 51 | 0.09 |
| 56 | 0.10 |
| 33 | 0.035 |
| 35 | 0.042 |

In addition to reporting the absolute IC₅₀ value as measured above, relative IC₅₀ values (rIC₅₀) are determined by a ratio of the IC₅₀ measured for the test compound to that of an internal control (reference) stated for each assay.

### Example 3

### Determination of Log D Values and Polar Surface Area of Glycomimetic Compounds

Log D values and the polar surface area (PSA) of glycomimetic compounds was determined using Marvin software (ChemAxon, One Broadway, Cambridge, Mass. 02142, USA).

The log D option for the calculation is: log P Method, weighted; Method weighs, VG=1, KLOP=1, PHYS=1, User defined=0; Electrolyte concentration: Cl−, Na+, K+ concentration (mol/dm3)=0.1.

The PSA option for the calculation is: take major microspecies at pH 7.4.

### Example 4

### Oral Bioavailability of Compounds in Rat Absorption Model

Oral bioavailability of glycomimetic compounds is determined in a rat intestinal absorption assay. Two groups of rats (3 animals per group) each receive a glycomimetic compound at a dose of 12.5 mg/kg administered. To one group of animals, the compound is administered intravenously and to the second group the compound is administered intraduodenally. The animals are bled pre-administration and at 5 minutes, 15 min, 30 min, and at 1, 2, 4, 8, and 24 hours after administration of the compound. The blood samples are collected into tubes containing anticoagulant. Plasma is prepared from each blood sample and frozen. The samples are then analyzed by LC-MS/MS to determine the level of compound in the plasma. The kinetics are determined and Cmax is calculated.

The various embodiments described above can be combined to provide further embodiments. All U.S. patents, U.S. patent application publications, U.S. patent applications, non-U.S. patents, non-U.S. patent applications, and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim the following:

1. A compound having the following formula (I): 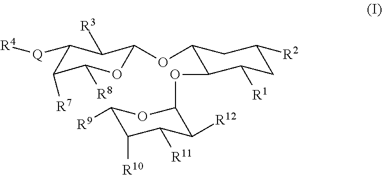
or a pharmaceutically acceptable salt, stereoisomer, tautomer, prodrug or solvate thereof, wherein:
Q is —O—, —S— or —CH₂—;
R¹ is C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₃-C₆ cycloalkyl, or C₃-C₆ halocycloalkyl;
R² is H, -L¹-C₁-C₂₅ alkyl, -L¹-C₂-C₂₅ alkenyl, -L¹-C₂-C₂₅ alkynyl, -L¹-C₁-C₂₅ haloalkyl, -L¹-C₂-C₂₅ haloalkenyl, -L¹-C₂-C₂₅ haloalkynyl or -L¹-M;
R³ is —OC(═O)aryl, —NHC(═O)R¹³ or -L-M;
R⁴ is aryl, aralkyl or has the following structure: 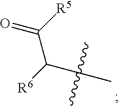
R⁵ is —OR¹⁴, —NHOR¹⁵, or —N(R¹⁵)(R¹⁶);
R⁶ is C₁-C₈ haloalkyl, C₂-C₈ haloalkenyl, C₂-C₈ haloalkynyl, cycloalkylalkyl or halocycloalkylalkyl;
R⁷, R¹⁰, R¹¹ and R¹² are each independently —OH, halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R⁸ is —CH₂OH, C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R⁹ and R¹³ are each independently C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R¹⁴ is H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl;
R¹⁵ and R¹⁶ are each independently H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl;
L¹ is an optional linker; and
M is a non-glycomimetic moiety,
wherein, the compound comprises at least one of the following:
Q is —S— or —CH₂—;
R¹ is C₂-C₈ alkenyl, C₂-C₈ alkynyl, C₁-C₈ haloalkyl, C₂-C₈ haloalkenyl, C₂-C₈ haloalkynyl, or C₃-C₆ halocycloalkyl;
R² is -L¹-C₂-C₂₅ alkenyl, -L¹-C₂-C₂₅ alkynyl, -L¹-C₁-C₂₅ haloalkyl, -L¹-C₂-C₂₅ haloalkenyl, -L¹-C₂-C₂₅ haloalkynyl or -L¹-M;
R³ is —NHC(═O)R¹³ or -L¹-M;
R₄ is aryl or aralkyl;
R₄ has the following structure: 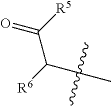
wherein R⁵ is —OR¹⁴ or —N(R¹⁵)(R¹⁶), wherein R¹⁴ is C₃-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl; or R⁶ is C₁-C₈ haloalkyl, C₂-C₈ haloalkenyl, C₂-C₈ haloalkynyl, cyclopropylalkyl, cyclobutylalkyl, cyclopentylalkyl or halocycloalkylalkyl;
at least one of R⁷, R¹⁰, R¹¹ or R¹² is independently halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R⁸ is C₁-C₈ alkyl, C₂-C₈ alkenyl, C₂-C₈ alkynyl, C₁-C₈ haloalkyl, C₂-C₈ haloalkenyl or C₂-C₈ haloalkynyl; or
R⁹ is C₂-C₈ alkenyl, C₂-C₈ alkynyl, C₁-C₈ haloalkyl, C₂-C₈ haloalkenyl or C₂-C₈ haloalkynyl.

2. The compound of claim 1, wherein the compound has the following structure (Ia): 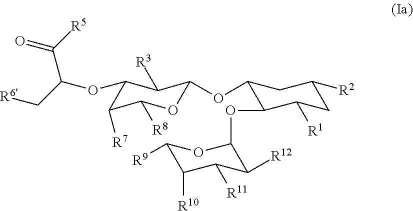
wherein R^{6′} is C₁-C₇ haloalkyl, C₂-C₇ haloalkenyl, C₂-C₇ haloalkynyl, cycloalkyl or halocycloalkyl.

3. The compound of claims 1 or 2, wherein at least one of R⁷, R¹⁰, R¹¹ or R¹² is —OH.

4. The compound of any one of the preceding claims, wherein R⁸ is —CH₂OH.

5. The compound of any one of the preceding claims, wherein R⁹ is methyl.

6. The compound of any one of the preceding claims, wherein R¹ is methyl or ethyl.

7. The compound of any one of the preceding claims, wherein the compound has the following structure (Ib):

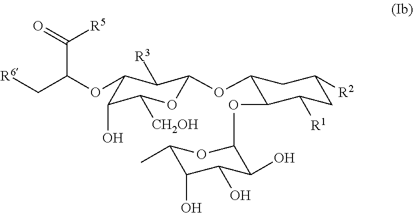

8. The compound of any one of claims 2-6, wherein R^{6′} is C₃-C₆ cycloalkyl.

9. The compound of claim 8, wherein R^{6′} is cyclopropyl or cyclohexyl, and the compound has one of the following structures (Ic) or (Id).

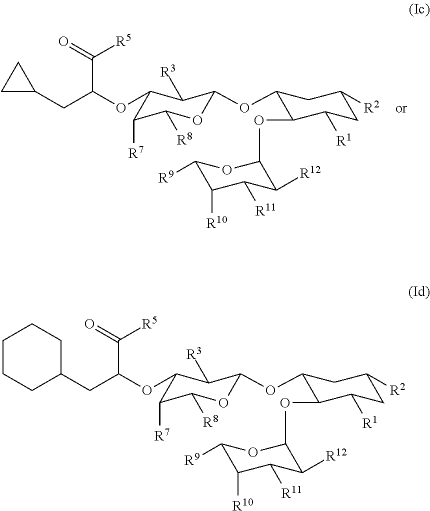

10. The compound of claim 1, wherein R⁶ is C₁-C₈ haloalkyl, C₂-C₈ haloalkenyl or C₂-C₈ haloalkynyl.

11. The compound of claim 10, wherein R⁶ is C₁-C₈ fluoroalkyl, C₂-C₈ fluoroalkenyl or C₂-C₈ fluoroalkynyl.

12. The compound of claim 11, wherein the compound has one of the following structures (Ie), (If) or (Ig): 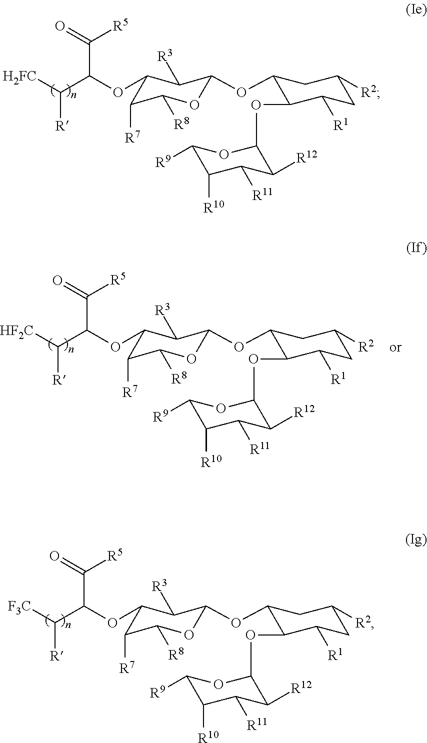
wherein:
R′ is, at each occurrence, independently H, halo, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl or C₃-C₆ halocycloalkyl group;
n is an integer from 1 to 7,
wherein R′ and n are selected such that R⁶ comprises no more than eight acyclic carbon atoms.

13. The compound of any one of claims 1-12, wherein R⁵ is —OH, —NHOCH₃, —NHOH, —N(CH₃)₂ or —NH(CHF₂).

14. The compound of claim 2, wherein the compound has the following structure (Ih): 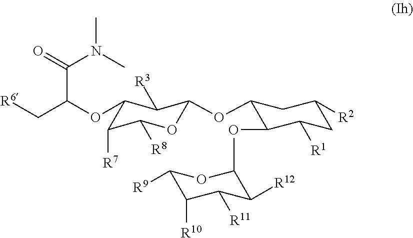
wherein R^{6′} is C₁-C₇ haloalkyl, C₂-C₇ haloalkenyl, C₂-C₇ haloalkynyl, C₃-C₆ cycloalkyl or C₃-C₆ halocycloalkyl.

15. The compound of any one of claims 1-12, wherein R⁵ is —OR¹³ and R¹³ is C₂-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl.

16. The compound of claim 15, wherein the compound has the following structure (Ii): 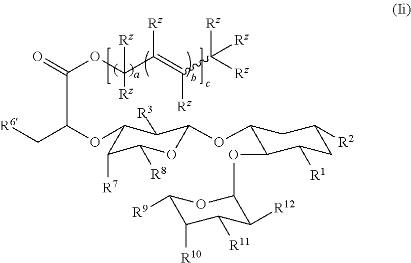
wherein:
R^{6′} is C₁-C₇ haloalkyl, C₂-C₇ haloalkenyl, C₂-C₇ haloalkynyl, C₃-C₆ cycloalkyl or C₃-C₆ halocycloalkyl;
R^{z} is, at each occurrence, independently H or halogen;
a and b are, at each occurrence, independently 0 or 1; and
c is an integer from 5 to 24,
wherein a, b and c are selected such that R¹³ comprises from 6 to 25 carbon atoms.

17. The compound of any one of claims 1-16, wherein R² is -L¹-C₁-C₂₅ alkyl, -L¹-C₂-C₂₅ alkenyl, -L¹-C₂-C₂₅ alkynyl, -L¹-C₁-C₂₅ haloalkyl, -L¹-C₂-C₂₅ haloalkenyl or -L¹-C₂-C₂₅ haloalkynyl.

18. The compound of claim 17, wherein the compound has the following structure (Ij): 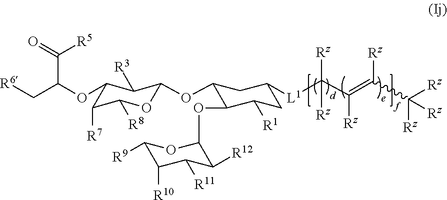
wherein:
R^{6′} is C₁-C₇ haloalkyl, C₂-C₇ haloalkenyl, C₂-C₇ haloalkynyl, C₃-C₆ cycloalkyl or C₃-C₆ halocycloalkyl;
R^{z} is, at each occurrence, independently H or halogen;
d and e are, at each occurrence, independently 0 or 1; and
f is an integer from 5 to 24,
wherein d, e and f are selected such that the alkyl or alkenyl moiety in R² comprises from 6 to 25 carbon atoms.

19. The compound of any one of claims 1-17, wherein R² is H.

20. The compound of any one of claims 1-17, wherein R² is -L¹-M.

21. The compound of any one of claims 1-17, wherein R³ is -L¹-M.

22. The compound of one of claims 20 or 21, wherein M is a steroidal moiety.

23. The compound of claim 22, wherein the steroidal moiety is cholic acid or a derivative thereof.

24. The compound of any one of claims 22 or 23, wherein M has the following structure (II): 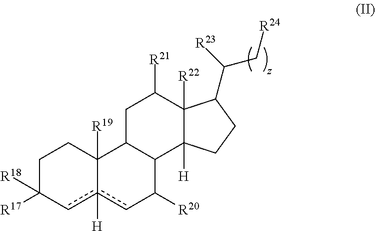
or a pharmaceutically acceptable salt, stereoisomer, tautomer, prodrug or solvate thereof, wherein:
R¹⁷ is H, —OH, —N₃, R²⁶, —N(R²⁷)(R²⁸), —NHC(═O)R²⁷, —C(═O)N(R²⁷)(R²⁸), —OC(═O)Ar, —NC(═O)Ar, —OC(═O)OR²⁹ or —OC(═O)R²⁹ or R¹⁷ joins with R¹⁸ to form oxo or ═NCH₂Ar;
R¹⁸ is H or —NH₂ or R¹⁸ joins with R¹⁷ to form oxo or ═NCH₂Ar;
R¹⁹, R²² and R²³ are each independently H, C₁-C₈ alkyl;
R²⁰ and R²¹ are each independently H, —OH, or R²⁶;
R²⁴ is R²⁶, —C(═O)OR³⁰, —CH₂OR²⁹, —C(═O)N(R³¹)(R³²), —C(═O)SR³⁰, —CH₂S(O)ₚ—SR³⁰, —CH₂N(R²⁷)(R²⁸) or —CH₂S(O)ₚ—SR³⁰;
R²⁶ is a direct bond to L¹ or a direct bond to a compound of structure (I);
R²⁷ and R²⁸ are each independently H, C₁-C₄ alkyl, C₃-C₇ cycloalkyl, C₄-C₁₀ alkylcycloalkyl or aryl or R²⁷ joins with R²⁸ to form a 4, 5, 6 or 7-membered heterocycle;
R²⁹ is H or C₁-C₃ alkyl;
R³⁰ is H C₁-C₁₀ alkyl, C₂-C₁₀ alkenyl, C₂-C₁₀ alkynyl, aryl or aralkyl;
R³¹ and R³² are each independently H, C₁-C₄ alkyl, C₃-C₇ cycloalkyl, C₄-C₁₀ alkylcycloalkyl, polyalkylamine or aryl or R³¹ joins with R³² to form a mono, bi or tricyclic heterocycle containing from 1 to 5 nitrogen atom;
Ar is optionally substituted phenyl;
p and z are each independently 0, 1 or 2; and
a dashed line indicates an optional double bond,
wherein all valences are satisfied.

25. The compound of claim 24, wherein the compound of structure (II) has one of the following structures (II′) or (II″):

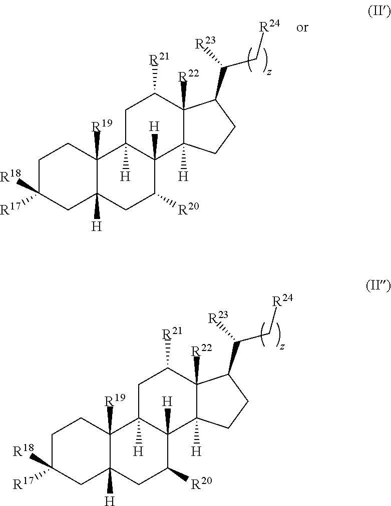

26. The compound of any one of claims 24 or 25, wherein R¹⁹, R²² and R²³ are each methyl.

27. The compound of any one of claims 24-26, wherein R¹⁸ is H.

28. The compound of any one of claims 24-26, wherein R¹⁸ is —NH₂.

29. The compound of any one of claims 24-28, wherein R¹⁷ is —OH.

30. The compound of any one of claims 24-29, wherein at least one of R²⁰ or R²¹ is H.

31. The compound of claim 30, wherein R²¹ is H.

32. The compound of any one of claims 24-31, wherein at least one of R²⁰ or R²¹ is —OH.

33. The compound of any one of claims 24-29, wherein each of R²⁰ and R²¹ is —OH.

34. The compound of any one of claims 24-33, wherein R²⁴ is —CO₂CH₃.

35. The compound of any one of claims 24-34, wherein at least one of R¹⁷, R¹⁸, R²¹ or R²⁴ is R²⁶.

36. The compound of claim 35, wherein at least two of R¹⁷, R²⁰, R²¹ or R²⁴ are R²⁶, such that the compound of structure (II) comprises two compounds of structure (I) covalently bound thereto.

37. The compound of any one of claims 1-36, wherein L¹ is present.

38. The compound of claim 37, wherein L¹ comprises methylene, ester, amide or ether functional groups or combinations thereof.

39. The compound of claim 38, wherein L¹ has one of the following structures: 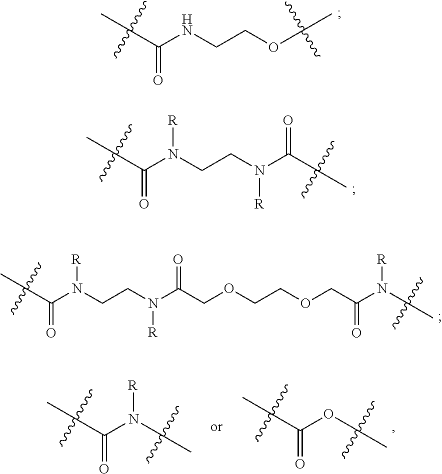
wherein each R is independently H or C₁-C₆ alkyl.

40. The compound of claim 1, wherein R³ is —NHC(═O)CH₃, —NHC(═O)CH₃, —OC(═O)phenyl or —OC(═O)cyclopropyl.

41. The compound of any one of claims 1-3, wherein R⁸ is C₁-C₈ haloalkyl.

42. The compound of claim 41, wherein R⁸ is —CH₂CHX₂.

43. The compound of claim 42, wherein X is F.

44. The compound of any one of claims 1-16, wherein R² is -L¹-C₁-C₈ haloalkyl or at least one of R¹, R⁷, R¹, R⁹, R¹², R¹³, R¹⁴, R¹⁵ or R¹⁶ is C₁-C₈ haloalkyl.

45. The compound of claim 44, wherein at least two of R¹, R⁷, R⁸, R⁹, R¹², R¹³, R¹⁴, R¹⁵ or R¹⁶ are C₁-C₈ haloalkyl.

46. The compound of any one of claims 44-45, wherein at least one C₁-C₈ haloalkyl is —CH₂(CH₂)ₘ—X, —(CH₂)ₘ—CHX₂, or —(CH₂)ₘ—CX₃, wherein m is an integer from 0 to 6 and X is F, Cl, Br or I.

47. The compound of claim 46, wherein X is F.

48. The compound of any one of claims 1-47, wherein R¹ is C₁-C₈ haloalkyl.

49. The compound of claim 48, wherein R¹ is —CH₂CHX₂.

50. The compound of claim 49, wherein X is F.

51. The compound of claim 1, wherein R⁴ is aryl or aralkyl.

52. The compound of claim 51, wherein R⁴ has one of the following structures: 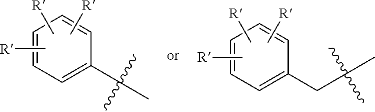
wherein each R′ is independently halo or hydroxyl.

53. The compound of claim 52, wherein R⁴ has one of the following structures:

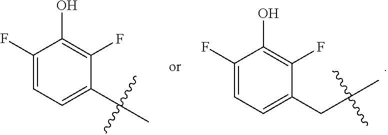 [SMILES: *c1ccc(F)c(O)c1F ; *Cc1ccc(F)c(O)c1F]

54. The compound of claim 1, wherein the compound has one of the following structures: 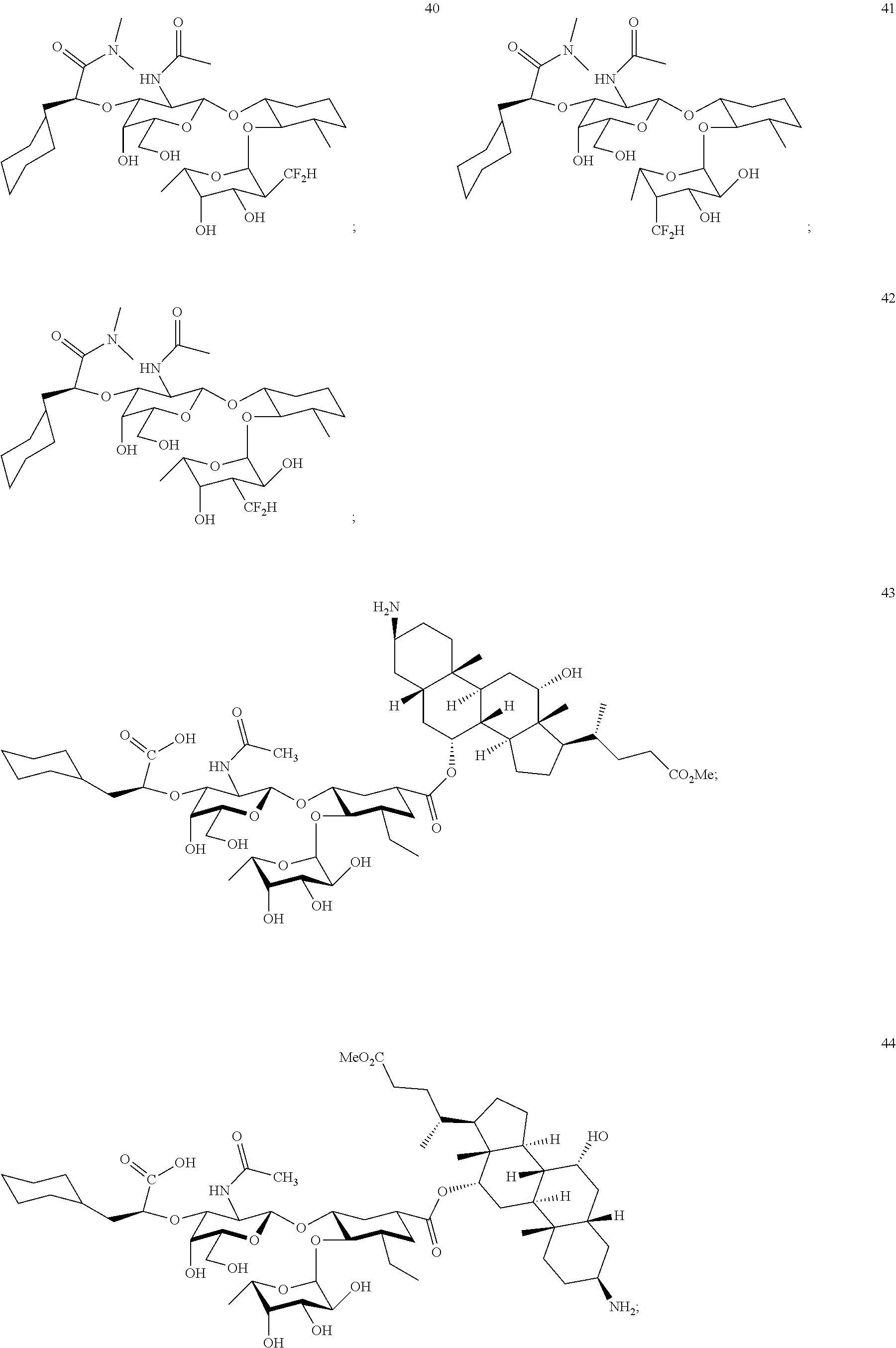 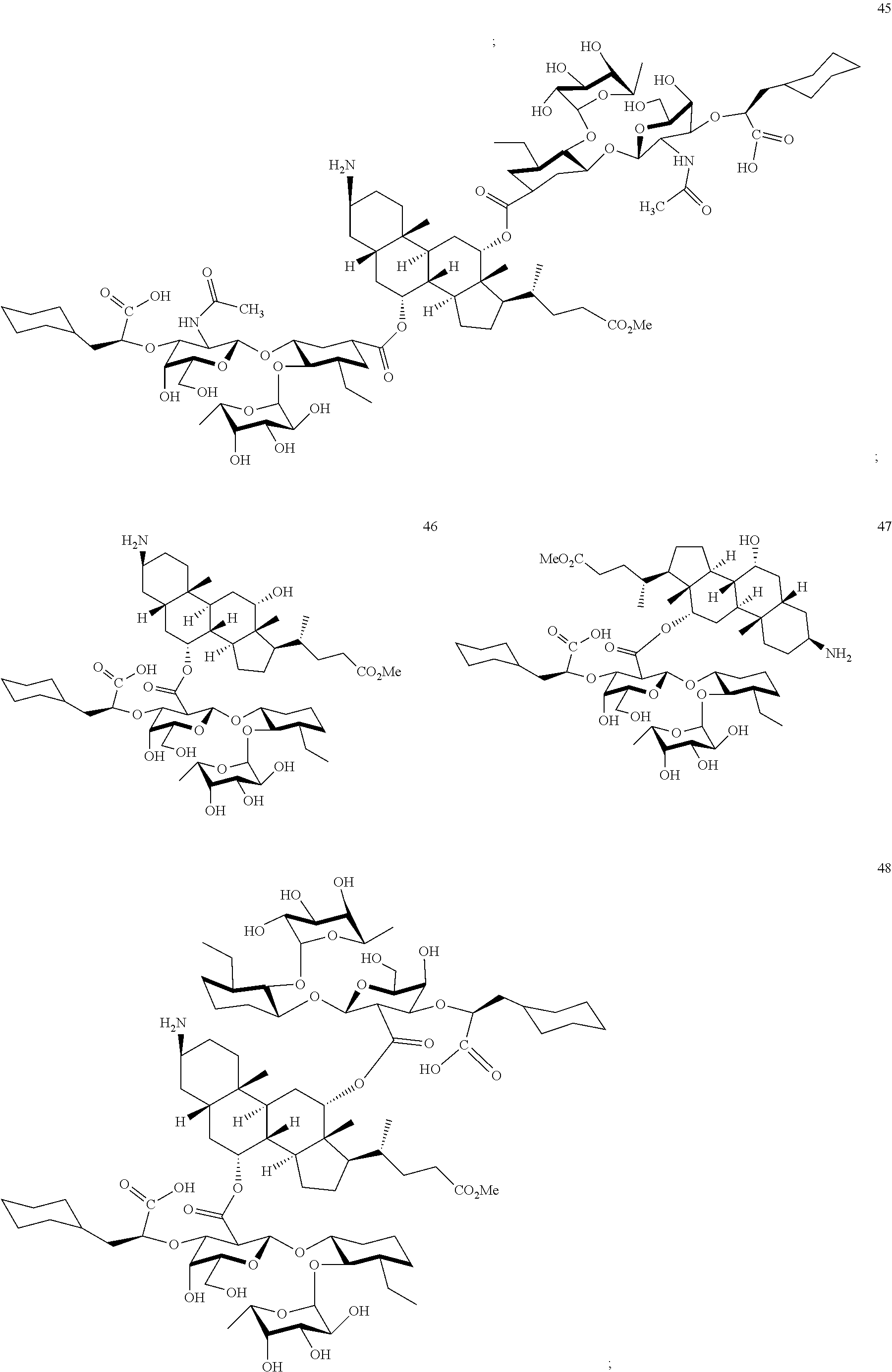 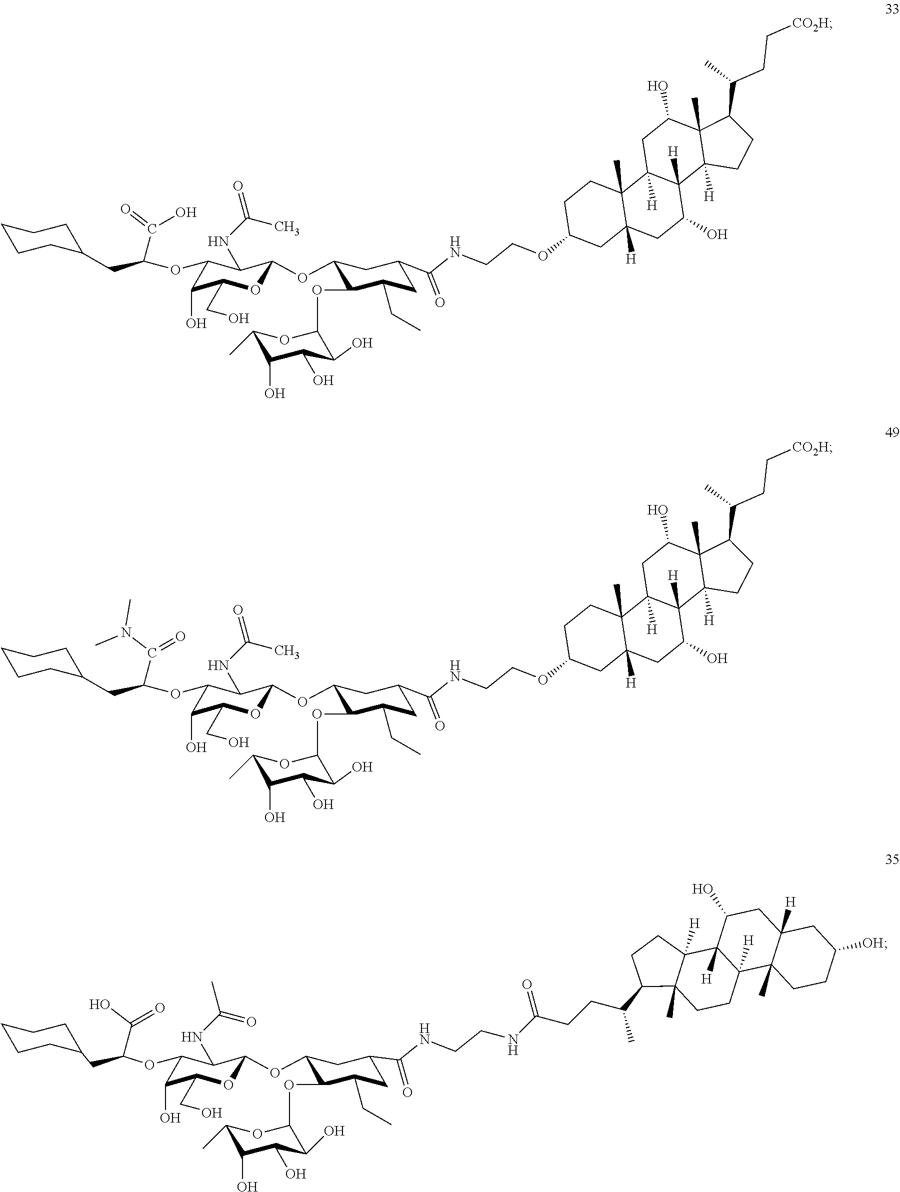 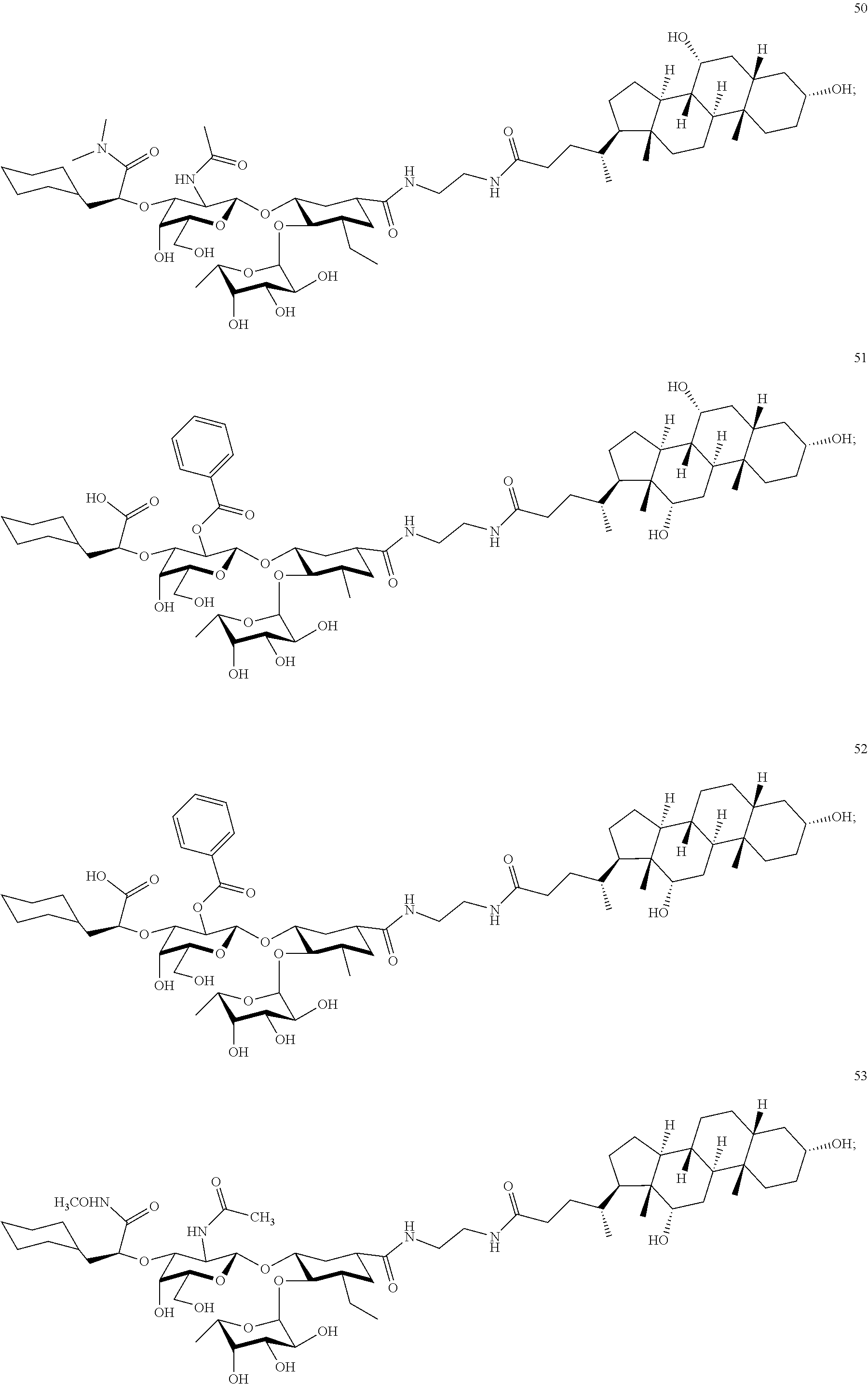 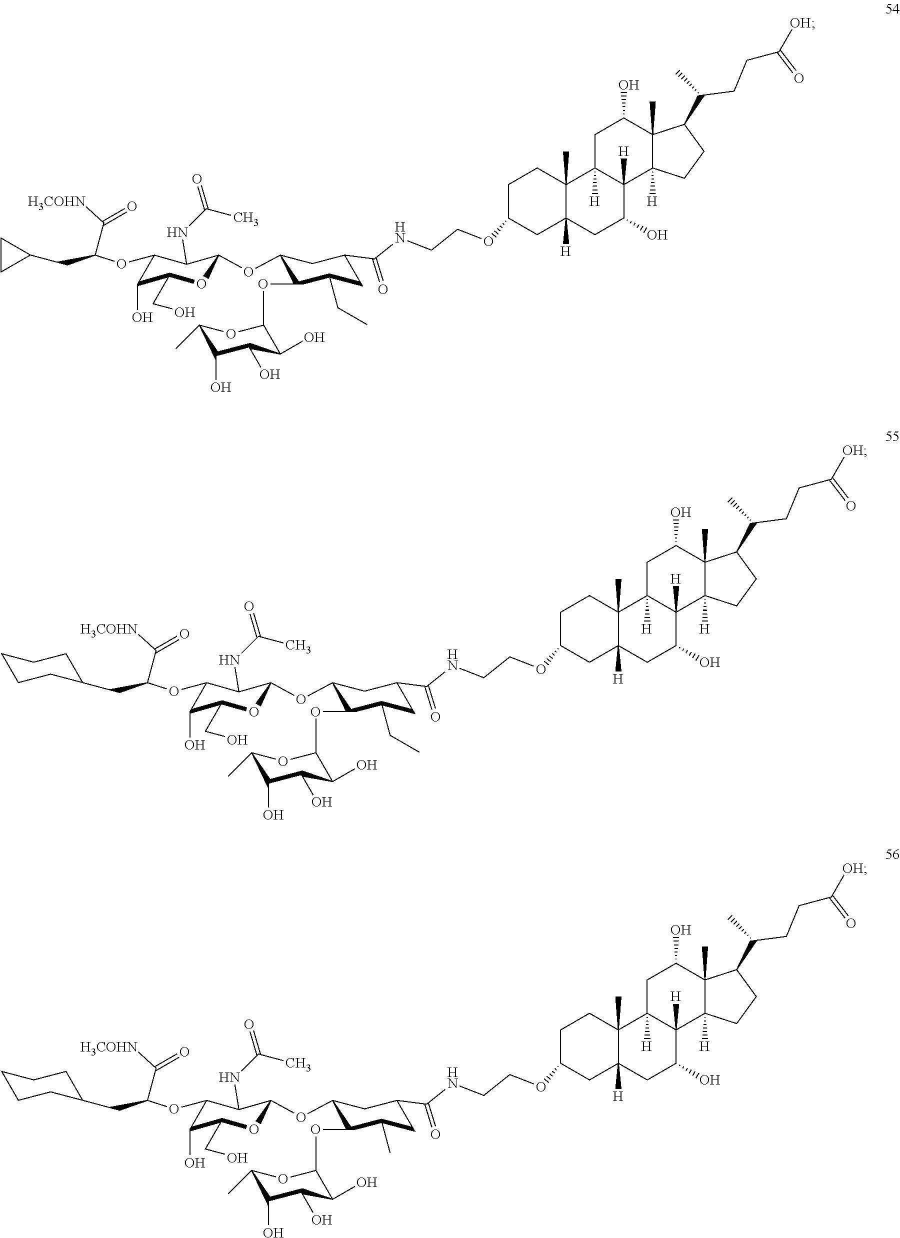 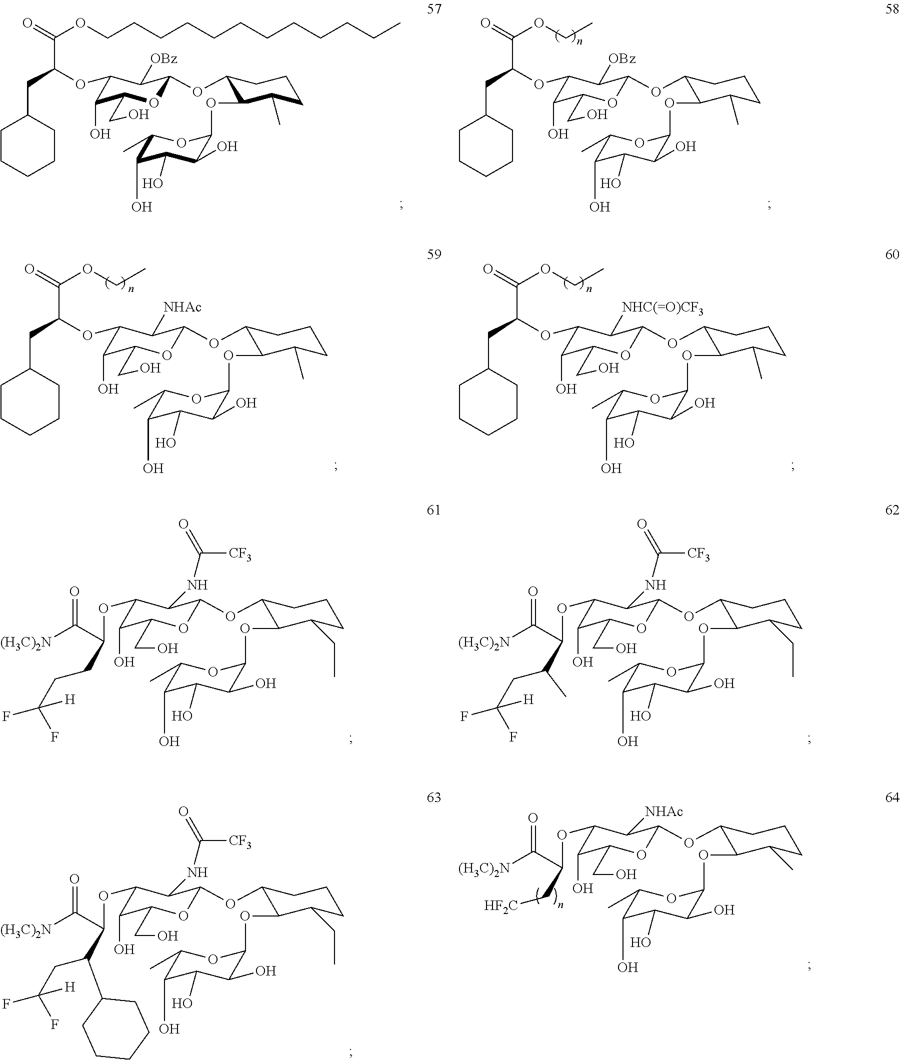 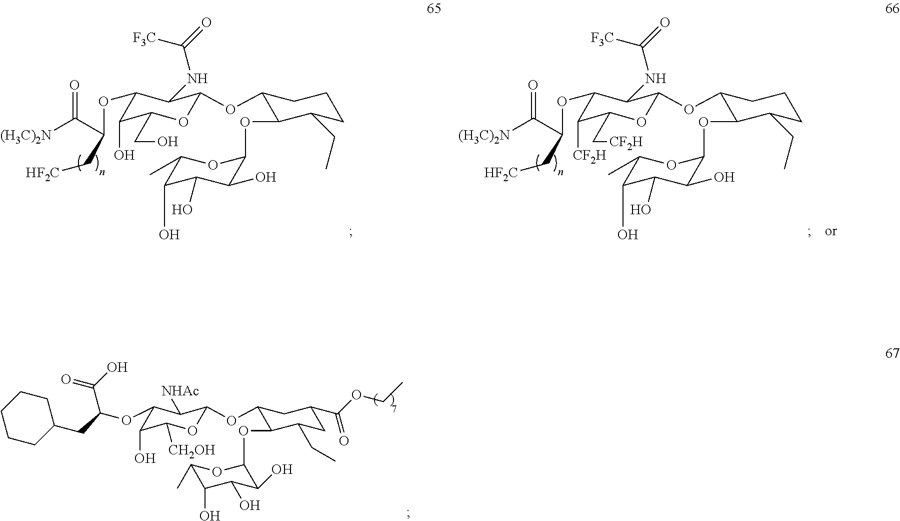
wherein n is an integer from 1-8.

55. The compound of claim 1, wherein Q is —O—.

56. A composition comprising the compound of any of claims 1-56 and a pharmaceutically acceptable carrier, diluent or excipient.

57. A composition comprising a compound of formula (Ik), a compound of formula (IId) and a pharmaceutically acceptable carrier, diluent or excipient, wherein the compound of formula (Ik) has the following structure: 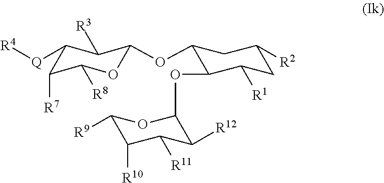
or a pharmaceutically acceptable salt, stereoisomer, tautomer, prodrug or solvate thereof, wherein:
Q is —O—, —S— or —CH₂—;
R¹ is C₁-C₈ alkyl, C₂-C₈ alkenyl, C₂-C₈ alkynyl, C₁-C₈ haloalkyl, C₂-C₈ haloalkenyl, C₂-C₈ haloalkynyl, C₃-C₆ cycloalkyl, or C₃-C₆ halocycloalkyl;
R² is H, -L¹-C₁-C₂₅ alkyl, -L¹-C₂-C₂₅ alkenyl, -L¹-C₂-C₂₅ alkynyl, -L¹-C₁-C₂₅ haloalkyl, -L¹-C₂-C₂₅ haloalkenyl or -L¹-C₂-C₂₅ haloalkynyl;
R³ is —OC(═O)aryl or —NHC(═O)R³;
R⁴ is aryl, aralkyl or has the following structure: 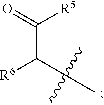
R⁵ is —OR¹⁴, —NHOR¹⁵, or —N(R¹⁵)(R¹⁶);
R⁶ is C₁-C₈ haloalkyl, C₂-C₈ haloalkenyl, C₂-C₈ haloalkynyl, cycloalkylalkyl or halocycloalkylalkyl;
R⁷, R¹⁰, R¹¹ and R¹² are each independently —OH, halo, C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R⁸ is —CH₂OH, C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R⁹ and R¹³ are each independently C₁-C₈ alkyl, C₂-C₈ alkenyl, C₂-C₈ alkynyl, C₁-C₈ haloalkyl, C₂-C₈ haloalkenyl or C₂-C₈ haloalkynyl;
R¹⁴ is H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl;
R¹⁵ and R¹⁶ are each independently H, C₁-C₂₅ alkyl, C₂-C₂₅ alkenyl, C₂-C₂₅ alkynyl, C₁-C₂₅ haloalkyl, C₂-C₂₅ haloalkenyl or C₂-C₂₅ haloalkynyl; and
L¹ is an optional linker,
and the compound of formula (IId) has the following structure: 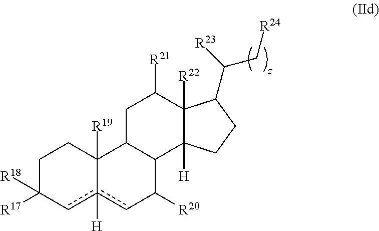
or a pharmaceutically acceptable salt, stereoisomer, tautomer, prodrug or solvate thereof, wherein:
R¹⁷ is H, —OH, —N₃, —OR²⁵, —N(R²⁷)(R²⁸), —NHC(═O)R²⁷, —C(═O)N(R²⁷)(R²⁸), —OC(═O)Ar, —NC(═O)Ar, —OC(═O)OR²⁹ or —OC(═O)R²⁹ or R¹⁷ joins with R¹⁸ to form oxo or ═NCH₂Ar;
R¹⁸ is H or —NH₂ or R¹⁸ joins with R¹⁷ to form oxo or ═NCH₂Ar;
R¹⁹, R²² and R²³ are each independently H, C₁-C₈ alkyl;
R²⁰ and R²¹ are each independently H, —OH or —OR²⁵;
R²⁴ is —C(═O)OR³⁰, —CH₂OR²⁹, —C(═O)N(R³¹)(R³²), —C(═O)SR³⁰, —CH₂S(O)ₚ—SR³⁰, —CH₂N(R²⁷)(R²⁸) or —CH₂S(O)ₚ—SR³⁰;
R²⁵ is a monosaccharide or an oligosaccharide comprising from 2-10 monosaccharides, wherein each glycosidic linkage at any anomeric carbon in the monosaccharide or oligosaccharide independently has the alpha or beta configuration;
R²⁷ and R²⁸ are each independently H, C₁-C₄ alkyl, C₃-C₇ cycloalkyl, C₄-C₁₀ alkylcycloalkyl or aryl or R²⁷ joins with R²⁸ to form a 4, 5, 6 or 7-membered heterocycle;
R²⁹ is H or C₁-C₃ alkyl;
R³⁰ is H C₁-C₁₀ alkyl, C₂-C₁₀ alkenyl, C₂-C₁₀ alkynyl, aryl or aralkyl;
R³¹ and R³² are each independently H, C₁-C₄ alkyl, C₃-C₇ cycloalkyl, C₄-C₁₀ alkylcycloalkyl, polyalkylamine or aryl or R³¹ joins with R³² to form a mono, bi or tricyclic heterocycle containing from 1 to 5 nitrogen atom;
Ar is optionally substituted phenyl;
p and z are each independently 0, 1 or 2; and
a dashed line indicate an optional double bond,
wherein all valences are satisfied.

58. The composition of claim 57, wherein the compound of structure (Ik) has the following structure (II): 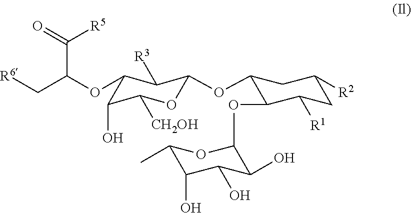
wherein R^{6′} is C₁-C₇ haloalkyl, C₂-C₇ haloalkenyl, C₂-C₇ haloalkynyl, C₃-C₆ cycloalkyl or C₃-C₆ halocycloalkyl.

59. The composition of any one of claims 57 or 58, wherein R¹⁷ is —OH, R¹⁸ is H and R²⁴ is —CO₂H.

60. The composition of any one of claims 57 or 58, wherein R¹⁷ is —OH, R¹⁸ is H and R²⁴ is —CO₂CH₃.

61. The composition any one of claims 57 or 58, wherein R¹⁸ is H, R¹⁹ is H and R²⁴ is —CO₂H.

62. The composition of any one of claims 57 or 58, wherein R¹⁷ is H, R¹⁸ is —NH₂ and R²⁴ is —CO₂CH₃.

63. The composition of any one of claims 57-62, wherein at least one of R¹⁷, R²⁰ or R²¹ is —OR²⁵.

64. The composition of any one of claims 57-63, wherein each of R²⁰ and R²¹ is —OR²⁵.

65. The composition of any one of claims 57-64, wherein R²⁵ is alpha or beta glucose.

66. A method for decreasing the likelihood of occurrence of metastasis of cancer cells in a subject in need thereof, the method comprising administering to the subject the pharmaceutical composition of any of claims 56-65.

67. A method for decreasing the likelihood of occurrence of infiltration of cancer cells into bone marrow in a subject in need thereof, the method comprising administering to the subject the pharmaceutical composition of any of claims 56-65.

68. A method for inhibiting adhesion of a tumor cell that expresses a ligand of E-selectin to an endothelial cell expressing E-selectin, the method comprising contacting the endothelial cell with a pharmaceutical composition comprising (a) a pharmaceutically acceptable excipient and (b) the compound of any one of claims 1-55, permitting the compound to interact with E-selectin present on the endothelial cell, thereby inhibiting binding of the tumor cell to the endothelial cell.

69. The method of claim 68, wherein the endothelial cell is present in the bone marrow.

70. A method for treating a cancer in a subject, the method comprising administering to the subject (a) the compound of any one of claims 1-55 or the pharmaceutical composition of any of claims 56-65 and (b) at least one of (i) chemotherapy and (ii) radiotherapy.

71. A method for decreasing the likelihood of occurrence of thrombus formation in a subject, comprising administering to the subject the compound of any one of claims 1-55 or the pharmaceutical composition of any one of claims 56-65.

72. A method for enhancing hematopoietic stem cell survival in a subject, comprising administering to the subject the compound of any one of claims 1-55 or the pharmaceutical composition of any one of claims 56-65.

73. The method of claim 72 wherein the subject has received or will receive chemotherapy or radiotherapy or both chemotherapy and radiotherapy.

74. The method of claim 73 wherein the subject has received or will receive two or more cycles of chemotherapy or radiotherapy.
